(19) United States
(12) Patent Application Publication
(10) Pub. No.: US 2020/0340009 A1
(43) Pub. Date: Oct. 29, 2020

(54) CENH3 DELETION MUTANTS

(71) Applicant: The Regents of the University of California, Oakland, CA (US)

(72) Inventors: Mily RON, Davis, CA (US); Sundaram KUPPU, Davis, CA (US); Anne B. BRITT, Davis, CA (US)

(21) Appl. No.: 16/958,126

(22) PCT Filed: Jan. 8, 2019
(86) PCT No.: PCT/US2019/012637

§ 371 Date: Jun. 25, 2020

Related U.S. Application Data
(60) Provisional application No. 62/614,867, filed on Jan. 8, 2018.

(51) Int. Cl. C12N 15/82; C12N 15/113; C12N 9/22; C07K 14/415

(52) U.S. Cl. CPC C12N 15/8287; C12N 15/8213; C12N 15/113; C12N 2800/80; C07K 14/415; C12N 2310/20; C12N 9/22

(57) ABSTRACT

Disclosed are methods of creating a haploid inducing plant by editing a CENH3 gene of a plant such that the CENH3 gene encodes a CENH3 polypeptide with a two or more contiguous amino acid deletion relative to wild-type CENH3, wherein said haploid inducing plant, when crossed with a second plant, results in haploid progeny. Also provided is a method of creating a haploid inducing plant by editing a CENH3 gene of a plant such that the CENH3 gene encodes a CENH3 polypeptide with a two or more contiguous amino acid insertion relative to wild-type CENH3, wherein said haploid inducing plant, when crossed with a second plant, results in haploid progeny.

### CROSS-REFERENCE TO RELATED PATENT APPLICATIONS

The present patent application claims benefit of priority to U.S. Provisional Patent Application No. 62/614,867, filed Jan. 8, 2018, which is incorporated by reference for all purposes.

### SEQUENCE LISTING

The instant application contains a Sequence Listing which has been submitted electronically in ASCII format and is hereby incorporated by reference in its entirety. Said ASCII copy, created on Jan. 4, 2019, is named 081906-1120833-228110PC_SL.txt and is 179,104 bytes in size.

### BACKGROUND OF THE INVENTION

Typical breeding of diploid plants relies on screening numerous plants to identify novel, desirable characteristics. Large numbers of progeny from crosses often must be grown and evaluated over several years in order to select one or a few plants with a desired combination of traits. Hybrid crops are generally produced as the immediate progeny of a cross between two inbred lines. These hybrids express exceptional characteristics derived from both parental genomes, but cannot be further propagated, as the various beneficial alleles segregate during meiosis, resulting in the loss of many of the hybrid's beneficial traits in the next generation. The production of hybrids relies on the production of elite true-breeding parental lines, each homozygous at all loci. These true-breeding lines are usually produced through the repeated self-pollination of an original more heterozygous stock, and are referred to as inbred lines. The production of these elite inbreds normally requires several generations.

The plant breeding process can be accelerated by producing haploid plants, the chromosomes of which can be doubled using colchicine or other means. Such doubled haploids produce homozygous lines in a single generation, which is significantly shorter than the approximately 8-10 generations of inbreeding that is typically required for diploid breeding. Thus, methods of producing haploid plants that can be doubled to generate fertile doubled haploids can dramatically improve the efficiency and effectiveness of plant breeding by producing true-breeding (homozygous) lines in only one generation.

Certain methods of inducing haploid plants by manipulating CENH3 have been described. For example, U.S. Pat. No. 8,618,354 describes introducing recombinant “tailswap” CENH3 constructs into a cenh3 plant to generate a plant (for ease of discussion referred to as a “haploid inducer”) that can be crossed to a second plant to generate progeny that had one set of chromosomes derived from the second plant, with no chromosomes derived from the haploid inducer. For example, if the second plant was diploid, at least some progeny of the cross would be haploid. PCT Publication No. WO2014/110274 describes generating haploid inducer plants by expressing a native CENH3 protein from one species in a different plant species. Expression of the first species' CENH3 in the different species was sufficient to allow for apparently normal mitosis, but resulted in some generation of progeny with half the number of chromosomes of the parent plant crossed to the haploid inducer plant. PCT Publication WO2016/138021 describes CENH3 amino acid substitutions.

### BRIEF SUMMARY OF THE INVENTION

Methods of creating a haploid inducing plant are provided. In some embodiments, the methods comprise editing a CENH3 gene of a plant such that the CENH3 gene encodes a CENH3 polypeptide with a two or more contiguous amino acid deletion relative to wild-type CENH3, wherein said haploid inducing plant, when crossed with a second plant, results in haploid progeny. In some embodiments, the CENH3 polypeptide has 10-12 (e.g., an eleven) amino acid deletion relative to wild-type CENH3. In some embodiments, the CENH3 polypeptide has a 2-15 (e.g., 2-12) contiguous amino acid deletion relative to wild-type CENH3. In some embodiments, the CENH3 polypeptide has a 1-15 (e.g., 1-12) or 1-70 (e.g., 2-60, e.g., 30-50) contiguous amino acid deletion relative to wild-type CENH3. In some embodiments, the deletion is in or at least part of the deletion includes one or more amino acid from the alpha-N helix domain of the CENH3 polypeptide. In some embodiments, the CENH3 polypeptide comprises a sequence at least 70, 80, 90, or 95% identical to SEQ ID NO:1-50 or 101-126. In some embodiments, the CENH3 polypeptide comprises a sequence at least 70, 80, 90, or 95% identical to any one of SEQ ID NO: 101, 110, 116-117, or 126-144. In some embodiments, the CENH3 polypeptide comprises any of SEQ ID NO:101-126. In some embodiments, the CENH3 polypeptide comprises any one of SEQ ID NO: 101, 110, 116-117, or 126-144. In some embodiments, the plant is a tomato or potato plant or another species as described herein. In some embodiments, the editing occurs in situ in the plant. In some embodiments, the editing comprises introducing into the plant a Cas (e.g., Cas9) or Cpf1 protein or other RNA-guided nuclease (e.g., Cms1 or TALENs) and a guide RNA targeting a CENH3-coding sequence, thereby inducing the two or more contiguous amino acid deletion.

Also provided is a method of creating a haploid inducing plant, the method comprising editing a CENH3 gene of a plant such that the CENH3 gene encodes a CENH3 polypeptide with a two or more contiguous amino acid insertion relative to wild-type CENH3, wherein said haploid inducing plant, when crossed with a second plant, results in haploid progeny. In some embodiments, the CENH3 polypeptide has a 2-15 (e.g., 2-12) contiguous amino acid insertion relative to wild-type CENH3. In some embodiments, the insertion is in a alpha-N helix domain of the CENH3 polypeptide. In some embodiments, the CENH3 polypeptide comprises a sequence at least 90% identical to SEQ ID NO:1-50 or 101-126. In some embodiments, the CENH3 polypeptide comprises any of SEQ ID NO: 101, 110, 116-117, or 126-144. In some embodiments, the plant is a tomato or potato plant. In some embodiments, the editing occurs in situ in the plant. In some embodiments, the editing comprises introducing into the plant a Cas protein or Cpf1 protein and a guide RNA targeting a CENH3-coding sequence, thereby inducing the two or more contiguous amino acid insertion.

Also provided is a haploid-inducing plant expressing a mutant CENH3 polypeptide encoded by a CENH3 coding sequence, wherein the CENH3 coding sequence comprises an in-frame deletion or insertion of 6 or more contiguous nucleotides, relative to wildtype CENH3. In some embodiments, the plant is homozygous for the CENH3 coding sequence. In some embodiments, the in-frame deletion or insertion comprises 6-42 contiguous nucleotides of the wildtype CENH3 gene. In some embodiments, the in-frame deletion or insertion comprises 6-33 contiguous nucleotides of the wildtype CENH3 gene. In some embodiments, the in-frame deletion or insertion is in or at least part of the deletion includes one or more amino acid from a sequence encoding an alpha-N helix domain of the CENH3 polypeptide. In some embodiments, the mutant CENH3 polypeptide comprises a sequence at least 70, 80, 90, or 95% identical to one of SEQ ID NO:1-50 or 101-126. In some embodiments, the mutant CENH3 polypeptide comprises a sequence at least 70, 80, 90, or 95% identical to one of any one of SEQ ID NO: 101, 110, 116-117, or 126-144. In some embodiments, the mutant CENH3 polypeptide comprises any of SEQ ID NO:101-126. In some embodiments, the mutant CENH3 polypeptide comprises any of SEQ ID NO: 101, 110, 116-117, or 126-144. In some embodiments, the plant is a tomato or potato plant.

Also provided is a method of making progeny with reduced chromosome content. In some embodiments, the method comprises crossing the plant as described above or elsewhere herein to a plant having a ploidy; and selecting progeny from the cross that have half the polidy. In some embodiments, wherein the plant has 2N chromosomes and the selected progeny have N chromosomes. In some embodiments, the progeny from the cross that have N chromosomes are haploid. In some embodiments, the plant is a tomato or potato plant.

### Definitions

“Centromeric histone H3” or “CENH3” refers to the centromere-specific histone H3 variant protein (also known as CENP-A). CENH3 is characterized by the presence of a highly variable N-terminal tail domain, which does not form a rigid secondary structure, and a conserved histone fold domain made up of three α-helical regions connected by loop sections. CENH3 is a member of the kinetochore complex, the protein structure on chromosomes where spindle fibers attach during cell division, and is required for kinetochore formation and for chromosome segregation.

An “endogenous” gene or protein sequence, as used with reference to an organism, refers to a gene or protein sequence that is naturally occurring in the genome of the organism.

A polynucleotide or polypeptide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “promoter,” as used herein, refers to a polynucleotide sequence capable of driving transcription of a coding sequence in a cell. Thus, promoters can include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “plant promoter” is a promoter capable of initiating transcription in plant cells. A “constitutive promoter” is one that is capable of initiating transcription in nearly all tissue types, whereas a “tissue-specific promoter” initiates transcription only in one or a few particular tissue types.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid, and hemizygous.

A “transgene” is used as the term is understood in the art and refers to a heterologous nucleic acid introduced into a cell by human molecular manipulation of the cell's genome (e.g., by molecular transformation). Thus, a “transgenic plant” is a plant that carries a transgene, i.e., is a genetically-modified plant. The transgenic plant can be the initial plant into which the transgene was introduced as well as progeny thereof whose genomes contain the transgene. In some embodiments, a transgenic plant is transgenic with respect to the CENH3 gene. In some embodiments, a transgenic plant is transgenic with respect to one or more genes other than the CENH3 gene.

The phrase “nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read through by a polymerase, and/or formation of double-stranded duplexes, and do not significantly alter expression of a polypeptide encoded by that nucleic acid.

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences which may be introduced to provide codon preference in a specific host cell.

The terms “identical” or percent “identity,” in the context of two or more nucleic acids or polypeptide sequences, refer to two or more sequences or subsequences that are the same or have a specified percentage of nucleotides or amino acid residues that are the same, when compared and aligned for maximum correspondence over a comparison window, as measured using one of the following sequence comparison algorithms or by manual alignment and visual inspection. Two nucleic acid sequences or polypeptides are said to be “identical” if the sequence of nucleotides or amino acid residues, respectively, in the two sequences is the same when aligned for maximum correspondence as described below. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

The phrase “substantially identical,” used in the context of two nucleic acids or polypeptides, refers to a sequence that has at least 50% sequence identity with a reference sequence (e.g., any one of SEQ ID NOs: 1-50 or 101-126 or SEQ ID NO: 127-144). Alternatively, percent identity can be any integer from 50% to 100%. Some embodiments include at least: 50%, 55%, 60%, 65%, 70%, 75%, 80%, 85%, 90%, 91%, 92%, 93%, 94%, 95%, 96%, 97%, 98%, or 99%, compared to a reference sequence using the programs described herein; preferably BLAST using standard parameters, as described below.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

An “expression cassette” refers to a nucleic acid construct that, when introduced into a host cell, results in transcription and/or translation of an RNA or polypeptide, respectively.

The phrase “host cell” refers to a cell from any organism. Exemplary host cells are derived from plants, bacteria, yeast, fungi, insects or other animals. Methods for introducing polynucleotide sequences into various types of host cells are known in the art.

A “mutated CENH3 polypeptide” refers to a CENH3 polypeptide that is a non-naturally-occurring variant from a naturally-occurring (i.e., wild-type) CENH3 polypeptide. As used herein, a mutated CENH3 polypeptide comprises one, two, three, four, or more amino acid deletions (and optionally also 1, 2, 3 or more amino acid additions or changes) relative to a corresponding wild-type CENH3 polypeptide (e.g., including but not limited to any of SEQ ID NOs: 1-50) while retaining the ability of the polypeptide to support mitosis and meiosis in a plant that does not express another CENH3 polypeptide. In this context, a “mutated” polypeptide can be generated by any method for generating non-wild type nucleotide sequences. In some embodiments, a mutated CENH3 polypeptide, when the only CENH3 polypeptide expressed in a plant, causes the plant to be a haploid inducer plant, meaning when the plant is crossed to a second plant, at least 0.1% of progeny have chromosomes only from the second plant.

An “amino acid deletion” refers to deleting one or more of the naturally occurring amino acid residue in a given position (e.g., the naturally occurring amino acid residue that occurs in a wild-type CENH3 polypeptide) such that the endogenous amino acids adjacent to the deleted amino acids are linked. For example, the naturally occurring amino acid residue at position 83 of the wild-type Arabidopsis CENH3 polypeptide sequence (SEQ ID NO:10) is glycine (G83); accordingly, an amino acid deletion at G83 refers to deleting the naturally occurring glycine such that amino acids P82 and T84 are joined without an intervening amino acid. One need not delete the amino acid from the protein, and instead may achieve the deletion by recombinant DNA technology. For example, deletion can be achieved by generation of recombinant DNA that codes for protein lacking the deleted amino acid.

An amino acid residue “corresponding to an amino acid residue [X] in [specified sequence]”, or an amino acid substitution “corresponding to an amino acid substitution [X] in [specified sequence]” refers to an amino acid in a polypeptide of interest that aligns with the equivalent amino acid of a specified sequence. Generally, as described herein, the amino acid corresponding to a position of a specified CENH3 polypeptide sequence can be determined using an alignment algorithm such as BLAST. In some embodiments, “correspondence” of amino acid positions (e.g. those deleted) is determined by aligning to a region of the CENH3 polypeptide comprising SEQ ID NO:10. When a CENH3 polypeptide sequence differs from SEQ ID NO:10 (e.g., by deletion of two or more amino acids), it may be that a particular mutation (i.e., deletion) associated with haploid inducing activity of a CENH3 mutant will not be in the same position number as it is in SEQ ID NO:10. For example, amino acid position 49 of Arabidopsis CENH3 (SEQ ID NO:10) aligns with amino acid position 13 of S. lycopersicum CENH3 (SEQ ID NO:29), as can be readily illustrated in an alignment of the two sequences. In this example, amino acid position 49 in SEQ ID NO:10 “corresponds” to position 13 in SEQ ID NO:29.

### BRIEF DESCRIPTION OF THE DRAWINGS

FIG. 1: Alignment of histone fold domain of CENH3 across kingdoms. Numbers (top row) represent S. pombe amino acids, beginning with the first amino acid of the histone fold domain. Both human histone 3 (bottom row) and CenpA (the human homolog of CENH3, top row) are depicted. FIG. 1 discloses SEQ ID NOS 183-195, respectively, in order of appearance.

FIG. 2: The predicted crystal structure of AtCENH3. This predicted structure (generated via Phyre2) is based on known CENPA crystal structures. The positions of the 2 aa and 11 aa deletions are illustrated. Position of deletions in CENH3 (indicated by brackets); numbering and aa code reflect the Arabidopsis aa sequence. FIG. 2 discloses “TVALKERHFQ” as SEQ ID NO: 145.

FIG. 3: pMR303. This T-DNA vector delivers: citrine:tailswap, an M4 guide targeting CenH3, driven by a AtU6-26 promoter, Cas9 driven by a 2×35SΩ promoter and a selectable marker.

FIG. 4: Illustrating the position of the N-alpha helix in the nucleosome. The transition from the N-terminal domain and the alpha-N helix is the point at which the N-terminal loop emerges from the interior of the nucleosome, passing very close to the wrapped DNA.

FIG. 5 illustrates the exon map of the Arabidopsis CENH3 gene, with locations of some guide RNAs used to generate indels described in the Examples shown.

FIG. 6 illustrates the exon map of the tomato CENH3 gene, with a location of a guide RNA used to generate some indels described in the Examples shown.

FIG. 7 illustrates a T-DNA vector used to target CenH3 in Arabidopsis. This T-DNA vector delivers: Cas9 driven by the AtRPS5a promoter; a guide targeting AtCenH3, driven by the AtU6-26 promoter; AtOLE1 pro-AtOLE1-Citrine-NOSter expression cassette as fluorescent marker, and a selectable marker.

FIG. 8 illustrates a T-DNA vector used to target CenH3 in Tomato. This T-DNA vector delivers: Cas9 driven by the AtUBI10 promoter; a guide targeting S1CenH3, driven by the AtU6-26 promoter; AtOLE1pro-AtOLE1-Citrine-NOSter expression cassette as fluorescent marker, and a selectable marker.

### DETAILED DESCRIPTION OF THE INVENTION

Endogenous Centromeric histone H3 (CENH3) proteins are a well characterized class of proteins that are variants of histone H3 proteins. These specialized proteins, which are specifically associated with the centromere, are essential for proper formation and function of the kinetochore, a multiprotein complex that assembles at centromeres and links the chromosome to spindle microtubules during mitosis and meiosis. Cells that are deficient in CENH3 fail to localize kinetochore proteins and show strong chromosome segregation defects.

CENH3 proteins are characterized by a N-terminal variable tail domain and a C-terminal conserved histone fold domain made up of three α-helical regions connected by loop sections. The CENH3 histone fold domain is conserved between CENH3 proteins from different species. See, e.g., Torras-Llort et al., EMBO J. 28:2337-48 (2009). In contrast, the N-terminal tail domains of CENH3 are highly variable even between closely related species. Histone tail domains (including CENH3 tail domains) are flexible and unstructured, as shown by their lack of strong electron density in the structure of the nucleosome determined by X-ray crystallography (Luger et al., Nature 389(6648):251-60 (1997)). Additional structural and functional features of CENH3 proteins can be found in, e.g., Cooper et al., Mol Biol Evol. 21(9):1712-8 (2004); Malik et al., Nat Struct Biol. 10(11):882-91 (2003); Black et al., Curr Opin Cell Biol. 20(1):91-100 (2008); and Torras-Llort et al., EMBO J. 28:2337-48 (2009).

CENH3 proteins are widely found throughout eukaryotes, and a large number of CENH3 proteins have been identified. See, e.g. SEQ ID NOs: 1-50. It will be appreciated that the above list is not intended to be exhaustive and that additional CENH3 sequences are available from genomic studies or can be identified from genomic databases or by well-known laboratory techniques. For example, where a particular plant or other organism species CENH3 is not readily available from a database, one can identify and clone the organism's CENH3 gene sequence using primers, which are optionally degenerate, based on conserved regions of other known CENH3 proteins.

The inventors have discovered that introduction of nucleotide deletions or insertions in a number divisible by three (e.g., 6 and 33) in a wildtype CENH3 coding sequence results in a viable CENH3 allele, which when homozygous in a plant and crossed with a wildtype diploid plant, results in haploid progeny. See, e.g., SEQ ID Nos: 101, 110, 116-117, or 126-144. Accordingly, methods are provided for introducing deletions or insertions of six or more nucleotides from a CENH3 coding sequence to delete nucleotides in a contiguous multiple of three to cause deletion or insertion of two or more amino acids, e.g., 2, 3, 4, 5, 6, 7, 8, 9, 10, 11, 12, 13, 14, 15 or more amino acids. In other embodiments, methods are provided for introducing deletions of three nucleotides from a CENH3 coding sequence to cause deletion of one amino acid. In other embodiments, methods are provided for introducing one or more nucleotide to a coding seqyence to introduce one or more amino acid addition to the CENH3 protein sequence. Also provided are plants comprising introduced nucleotide deletions as discussed above or elsewhere herein. Methods of crossing such plants with a parent plant to generate a progeny plant having half the chromosomes of the parent plant are also provided.

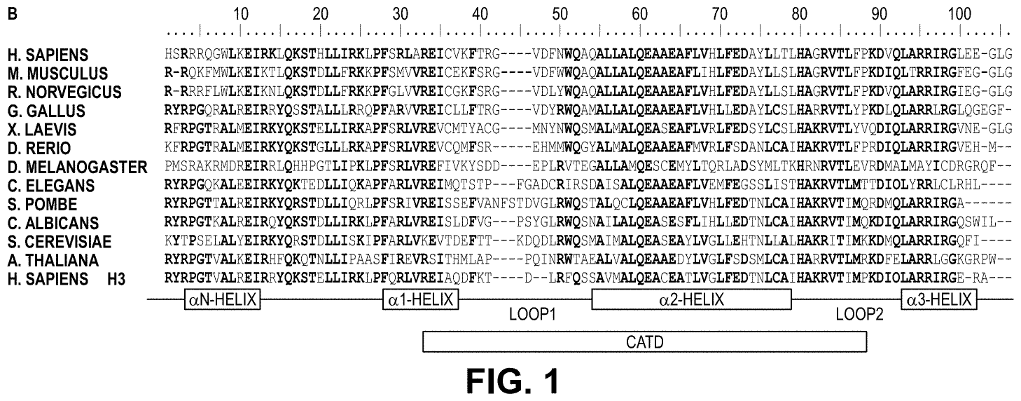

Deletions or insertions in the CENH3 polypeptides can occur at various locations. In some embodiments, the deletion is in or at least part of the deletion includes one or more amino acid from the histone-fold domain. For example, the deletion or insertion can include deletion of one or more (e.g., 2, 3, 4, 5, 6, 7, 8, 9, 10, 11, 12, 13, 14, 15 or more) contiguous amino acid in any of the alpha-N helix domain, alpha-1 helix domain, alpha-2 helix domain, or alpha-2 helix domain, and/or an intervening amino acid as occurs in the respective (e.g., most closely aligned and/or from which the deleted sequence has been derived) wildtype CENH3 polypeptide. These domains are shown for representative sequences in FIG. 1. To the extent the polypeptides are said to “comprise” a deletion this means that the polypeptide in question lacks those deleted amino acids as compared to the reference wildtype CENH3 sequence. In some embodiments, the deletion includes one or more contiguous amino acid corresponding to TVALKEIRHFQ (SEQ ID NO: 145), e.g., as occurs in tomato CENH3. In some embodiments, the deletion is in or at least part of the deletion includes one or more amino acid from the CENH3 tail domain. In some embodiments, the insertion occurs at an internal sequence of CENH3 (i.e., not at the amino or carboxyl terminus).

The CENH3 histone fold domain is conserved between CENH3 proteins from different species. The CENH3 histone fold domain can be distinguished by three α-helical regions connected by loop sections. While it will be appreciated that the exact location of the histone fold domain will vary in CENH3 proteins from other species, it will be found at the carboxyl terminus of an endogenous (wildtype) CENH3 protein. Thus, in some embodiments, a CENH3 protein can be identified in an endogenous protein as having a carboxyl terminal domain substantially similar (e.g., at least 30%, 40%, 50%, 60%, 70%, 85%, 90%, 95% or more identity) to any of SEQ ID NO:s 55-100.

The border between the tail domain and the histone fold domain of CENH3 proteins is at, within, or near (i.e., within 5, 10, 15, 20, or 25 amino acids from the “P” of) the conserved PGTVAL sequence (SEQ ID NO: 146). The PGTVAL sequence (SEQ ID NO: 146) is approximately 81 amino acids from the N terminus of the Arabidopsis CENH3 protein, though the distance from the N terminus of different endogenous CENH3 proteins varies. See, for example, the sequence listing.

Deletions as described herein (for example but not limited to those corresponding to the above-described positions) can be introduced into a CENH3 coding sequence from any species. In some embodiments the CENH3 polypeptide has one of the deletions described herein and is substantially identical to any one of SEQ ID NOs:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, the CENH3 that has an introduced deletion is from a species of plant of the genus Abelmoschus, Allium, Apium, Amaranthus, Arachis, Arabidopsis, Asparagus, Atropa, Avena, Benincasa, Beta, Brassica, Cannabis, Capsella, Cica, Cichorium, Citrus, Citrullus, Capsicum, Carthamus, Cocos, Coea, Cucumis, Cucurbita, Cynasa, Daucus, Diplotaxis, Dioscorea, Elais, Eruca, Foeniculum, Fragaria, Glycine, Gossypium, Helianthus, Heterocallis, Hordeum, Hyoscyamus, Ipomea, Lactuca, Lagenaria, Lepidium, Linum, Loliur, Luffa, Luula, Lycopersicon, Malus, Manihot, Majorana, Medicago, Momodica, Musa, Nicotiana, Olea, Oryza, Panicum, Pastinaca, Pennisetum, Persea, Petroselinium, Phaseolus, Physalis, Pinus, Pisum, Populus, Pyrus, Prunus, Raphanus, Saccharum, Secale, Senecio, Sesamum, Sinapis, Solanum, Sorghum, Spinacia, Theobroma, Trichosantes, Trigonella, Triticum, Turritis, Valerianelle, Vitis, Vigna, or Zea. For example, the CENH3 deletion can be in a tomato, potato, rice, Arabidopsis or other plant CENH3 and can be expressed in the same species or a different species of plant. The resulting deleted CENH3 polypeptide can be expressed in the same plant species from which the CENH3 polypeptide was derived or the CENH3 polypeptide having the deletion can be expressed in a different species.

Mutation methods that introduce DNA deletions, as well as site-directed mutagenesis can be used to generate the deletions described herein as desired. Methods for introducing genetic deletions into plant genes and selecting plants with desired traits are well known and can be used to introduce deletions into or to knock out the CENH3 gene. For instance, seeds or other plant material can be treated with a mutagenic insertional polynucleotide (e.g., transposon, T-DNA, etc.) or chemical substance, according to standard techniques. Chemical substances that cause deletions include, but are not limited to, bleomycin and nalidixic acid. Alternatively, ionizing radiation from sources such as, X-rays or gamma rays can be used. Plants having a mutated or knocked-out CENH3 gene can be identified, for example, by phenotype or by molecular techniques, including but not limited to TILLING methods. See, e.g., Comai, L. & Henikoff. S. The Plant Journal 45, 684-694 (2006).

CENH3 polypeptides having deletions as described herein can also be constructed in vitro by mutating the DNA sequences that encode the corresponding wild-type CENH3 polypeptide (e.g., a wild-type CENH3 polypeptide of any of SEQ ID NOs:1-50), such as by using site-directed or random mutagenesis. Nucleic acid molecules encoding the wild-type CENH3 polypeptide can be mutated in vitro to have one or more deletions by a variety of polymerase chain reaction (PCR) techniques. See, e.g., PCR Strategies (M. A. Innis, D. H. Gelfand, and J. J. Sninsky eds., 1995, Academic Press, San Diego, Calif.) at Chapter 14; PCR Protocols: A Guide to Methods and Applications (M. A. Innis, D. H. Gelfand, J. J. Sninsky, and T. J. White eds., Academic Press, NY, 1990).

As a non-limiting example, mutagenesis may be accomplished using site-directed mutagenesis, in which deletions are made to a DNA template. Kits for site-directed mutagenesis are commercially available, such as the QuikChange Site-Directed Mutagenesis Kit (Stratagene). Briefly, a DNA template to be mutagenized is amplified by PCR according to the manufacturer's instructions using a high-fidelity DNA polymerase (e.g., Pfu Turbo™) and oligonucleotide primers containing the desired mutation (e.g., deletion). Incorporation of the oligonucleotides generates a mutated plasmid, which can then be transformed into suitable cells (e.g., bacterial or yeast cells) for subsequent screening to confirm mutagenesis of the DNA.

Other mutation induction systems, such as genome editing methods, can be used to target deletions in CENH3 (Lozano-Juste, J., and Cutler, S. R (2014) Trends in Plant Science 19, 284-287). The sequence-specific introduction of a double stranded DNA break (DSB) in a genome leads to the recruitment of DNA repair factors at the breakage site, which then repair lesion by either the error-prone non-homologous end joining (NHEJ) or homologous recombination (HR) pathways. NHEJ repairs the breaks, but is imprecise and often creates diverse mutations at and around the DSB. In cells in which the HR machinery repairs the DSB, sequences with homology flanking the DSB, including exogenously supplied sequences, can be incorporated at the region of the DSB. DSBs can therefore be leveraged by geneticists to increase the frequency of mutations at defined sites, however intrinsic differences between the relative roles of HR and NHEJ can affect the mutation types at a targets locus. A number of technologies have been developed to create DSBs at specific sites including synthetic zinc finger nucleases (ZFNs), transcription activator-like endonucleases (TALENs) and most recently the clustered regularly interspaced short palindromic repeats (CRISPR)/CRISPR-associated protein 9 (Cas9) system. This system is based on a bacterial immune system against invading bacteriophages in which a complex of 2 small RNAs, the CRISPR-RNA (crRNA) and the trans-activating crRNA (tracrRNA) directs a nuclease (Cas9) to a specific DNA sequence complementary to the crRNA. In other embodiments, Cpf-1 or other Class 2 CRISPR proteins or CRISPR-associated protein (CAS) CRISPR-associated protein (e.g., other Class 1 CRISPR proteins) from other bacteria, for example, can be similarly used. Using any of these systems, one can create DSBs at pre-determined sites in cells expressing the genome editing constructs. In order for homologous recombination to occur, a DNA cassette homologous to the targeted site must be provided, preferably at a high concentration so that homologous recombination is favored or NHEJ. Multiple strategies are conceivable for realizing this, including template delivery using agrobacterium mediated transformation or particle bombardment of DNA templates, and one recently described method uses a modified viral genome to provide the double stranded DNA template. For example, Baltes et al. 2014 (Baltes, N. J., et al. (2014) Plant Cell 26, 151-163) recently demonstrated that an engineered geminivirus that was introduced into plant cells using Agrobacterium mediated transformation could be engineered to produce DNA recombination templates in cells where a ZFN was co-expressed.

In the CRISPR/Cas9 bacterial antiviral and transcriptional regulatory system, a complex of two small RNAs—the CRISPR-RNA (crRNA) and the trans-activating crRNA (tracrRNA)—directs the nuclease (Cas9) to a specific DNA sequence complementary to the crRNA (Jinek, M., et al. Science 337, 816-821 (2012)). Binding of these RNAs to Cas9 involves specific sequences and secondary structures in the RNA. The two RNA components can be simplified into a single element, the single guide-RNA (sgRNA), which is transcribed from a cassette containing a target sequence defined by the user (Jinek, M., et al. Science 337, 816-821 (2012)). This system has been used for genome editing in humans, zebrafish, Drosophila, mice, nematodes, bacteria, yeast, and plants (Hsu, P. D., et al., Cell 157, 1262-1278 (2014)). In this system the nuclease creates double stranded breaks at the target region programmed by the sgRNA. These can be repaired by non-homologous recombination, which often yields inactivating mutations. The breaks can also be repaired by homologous recombination, which enables the system to be used for gene targeted gene replacement (Li, J.-F., et al. Nat. Biotechnol. 31, 688-691, 2013; Shan, Q., et al. Nat. Biotechnol. 31, 686-688, 2013). The CENH3 mutations described in this application can be introduced into plants using the CAS9/CRISPR or other CRISPR system.

Accordingly, in some embodiments, instead of generating a transgenic plant, a native CENH3 coding sequence in a plant or plant cell can be altered in situ to generate a plant or plant cell carrying a polynucleotide encoding a CENH3 polypeptide having one or more deletion as described herein. The CRISPR/Cas system has been modified for use in prokaryotic and eukaryotic systems for genome editing and transcriptional regulation. The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize the RNA-mediated nuclease, Cas9 in complex with guide and activating RNA to recognize and cleave foreign nucleic acid. Cas9 homologs are found in a wide variety of eubacteria, including, but not limited to bacteria of the following taxonomic groups: Actinobacteria, Aquficae, Bacteroidetes-Chlorobi, Chlamydiae-Verrucomicrobia, Chofiexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21. In some embodimemts, a Cms1 nuclease is used. See, e.g., Begemann, Matthew B., et al., bioRxiv (2017): 192799. Other exemplary nucleases include, for example, TALE nucleases (TALENs), zinc-finger proteins (ZFPs), zinc-finger nucleases (ZFNs), DNA-guided polypeptides such as Natronobacterium gregoryi Argonaute (NgAgo).

The present disclosure also provides for nucleic acids, including isolated nucleic acids, nucleic acid expression cassettes, and expression vectors, that encode the CENH3 polypeptides having one or more deletion as described herein. Also provided are cells comprising the nucleic acids.

Once a polynucleotide encoding a CENH3 polypeptide having the deletion(s) is obtained, in some embodiments, it can also be used to prepare an expression cassette for expressing the resulting modified CENH3 polypeptide in a transgenic plant, directed by a promoter, which can be endogenous (e.g., a CENH3 promoter) or heterologous. Expression of the CENH3 polynucleotides encoding the polypeptide having the deletion(s) in a genetic background that otherwise does not express other CENH3 proteins, is useful, for example, to make a haploid inducer plant.

Any of a number of means can be used to drive CENH3 (having a deletion as described herein) activity or expression in plants. In some embodiments, to use a polynucleotide sequence for a CENH3 polypeptide having a deletion in the above techniques, recombinant DNA vectors suitable for transformation of plant cells are prepared. Techniques for transforming a wide variety of higher plant species are well known and described in the technical and scientific literature. See, e.g., Weising et al. Ann. Rev. Genet. 22:421-477 (1988). A DNA sequence coding for the CENH3 polypeptide having a deletion can be combined with transcriptional and translational initiation regulatory sequences which will direct the transcription of the sequence from the gene in the intended tissues of the transformed plant.

For example, a plant promoter fragment may be employed to direct expression of the CENH3 polynucleotide having a deletion in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill.

Alternatively, the plant promoter may direct expression of the CENH3 protein having a deletion in a specific tissue (tissue-specific promoters) or may be otherwise under more precise environmental control (inducible promoters).

If proper protein expression is desired, a polyadenylation region at the 3′-end of the coding region should be included. The polyadenylation region can be derived from a naturally occurring CENH3 gene, from a variety of other plant genes, or from T-DNA.

In some embodiments, the vector comprising the sequences (e.g., promoters or CENH3 coding regions) comprises a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or Basta.

In some embodiments, the CENH3 nucleic acid sequence having a deletion is expressed recombinantly in plant cells. A variety of different expression constructs, such as expression cassettes and vectors suitable for transformation of plant cells, can be prepared. Techniques for transforming a wide variety of higher plant species are well known and described in the technical and scientific literature. See, e.g., Weising et al. Ann. Rev. Genet. 22:421-477 (1988). A DNA sequence coding for a CENH3 protein can be combined with cis-acting (promoter) and trans-acting (enhancer) transcriptional regulatory sequences to direct the timing, tissue type and levels of transcription in the intended tissues of the transformed plant. Translational control elements can also be used.

Embodiments of the present disclosure also provide for a mutated CENH3 nucleic acid operably linked to a promoter which, in some embodiments, is capable of driving the transcription of the CENH3 coding sequence having a deletion in plants. The promoter can be, e.g., derived from plant or viral sources. The promoter can be, e.g., constitutively active, inducible, or tissue specific. In construction of recombinant expression cassettes, vectors, transgenics, of the invention, different promoters can be chosen and employed to differentially direct gene expression, e.g., in some or all tissues of a plant or animal.

When generating transgenic plants, it will be desirable to ultimately generate a plant that expresses the CENH3 polypeptide having a deletion but does not express wildtype CENH3. In some embodiments, one can generate a CENH3 mutation in an endogenous gene that reduces or eliminates CENH3 activity or expression, e.g., generating a CENH3 gene knockout. In these embodiments, one can generate an organism heterozygous for the gene knockout or mutation and introduce an expression cassette for expression of the heterologous corresponding mutated kinetochore complex protein into the organism. Progeny from the heterozygote can then be selected that are homozygous for the mutation or knockout but that comprises the recombinantly expressed heterologous mutated kinetochore complex protein. Accordingly, in some embodiments, plants, plant cells or other organisms are provided in which one or both endogenous CENH3 alleles are knocked out or mutated to significantly or essentially completely lack CENH3 activity, i.e., sufficient to induce embryo lethality without a complementary expression of a mutated CENH3 protein as described herein. In plants having more than a diploid set of chromosomes (e.g. tetraploids), all alleles can be inactivated, mutated, or knocked out.

Alternatively, one can introduce the expression cassette encoding a CENH3 protein having a deletion into an organism with an intact set of endogenous CENH3 alleles and then silence the endogenous CENH3 gene. As an example, an siRNA or microRNA can be introduced or expressed in the organism that reduces or eliminates expression of the endogenous CENH3.

The silencing siRNA or other silencing agent can be selected to silence the endogenous CENH3 gene but not substantially interfere with expression of the CENH3 protein having a deletion. In situations where endogenous CENH3 is to be inactivated, this can be achieved, for example, by targeting the siRNA to the N-terminal tail coding section, or untranslated portions, or the CENH3 mRNA, depending on the structure of the mutated kinetochore complex protein. Alternatively, the CENH3 protein transgene having a deletion can be designed with novel codon usage, such that it lacks sequence homology with the endogenous CENH3 protein gene and with the silencing siRNA.

Also provided are host cell(s) comprising a nucleic acid encoding a CENH3 polypeptide having a deletion as described herein. As discussed above, the cell can comprise an endogenous CENH3 gene that has been mutated to contain the nucleic acid encoding the CENH3 polypeptide having a deletion, or the nucleic acid can be heterologous to the cell (for example, the nucleic acid could be transformed into the cell). In the latter case, the nucleic acid can be part of a heterologous expression cassette (e.g., comprising a promoter operably linked to the coding sequence). Exemplary host cells include, for example, prokaryotic (e.g., including but not limited to E. coli) cells or eukaryotic cells, and can for example plant, fungal, yeast, mammalian, insect, or other cells. Also provided as discussed above are plants comprising a nucleic acid encoding a CENH3 polypeptide having a deletion as described herein.

Crossing a plant that expresses a CENH3 polypeptide having a deletion as described herein, and that does not express a wildtype CENH3 polypeptide, either as a pollen or ovule parent, to a diploid plant that expresses an endogenous CENH3 polypeptide will result in at least some progeny (e.g., at least 0.1%, 0.5%, 1%, 5%, 10%, 20% or more) that are haploid and comprise only chromosomes from the plant that expresses the endogenous CENH3 polypeptide. Thus, the present disclosure allows for the generation of haploid plants having all of its chromosomes from a plant of interest (i.e., the plant expressing the endogenous CENH3 polypeptide) by crossing the plant of interest with a plant expressing the mutated CENH3 polypeptide and collecting and/or selecting the resulting haploid seed. The methods can similarly be used to generate plants with higher number of chromosomes to generate progeny with half the number of chromosomes, e.g., crossing a plant that expresses a CENH3 polypeptide having a deletion as described herein, and that does not express a wildtype CENH3 polypeptide to a tetraploid plant will generate some progeny that have half the chromosomes of the tetraploid plant (e.g., diploid plants).

As noted above, the plant expressing a wild type (e.g., endogenous) CENH3 protein can be crossed as either the male or female parent. An aspect of the method is that it allows for generation of a plant (or other organism) having only a male parent's nuclear chromosomes and a female parent's cytoplasm with associated mitochondria and plastids, when the mutated CENH3 polypeptide parent is the female parent.

Once generated, haploid plants can be used for a variety of useful endeavors, including but not limited to the generation of doubled haploid plants, which comprise an exact duplicate copy of chromosomes. Such doubled haploid plants are of particular use to speed plant breeding, for example. A wide variety of methods are known for generating doubled haploid organisms from haploid organisms.

Somatic haploid cells, haploid embryos, haploid seeds, or haploid plants produced from haploid seeds can be treated with a chromosome doubling agent. Homozygous double haploid plants can be regenerated from haploid cells by contacting the haploid cells, including but not limited to haploid callus, with chromosome doubling agents, such as colchicine, anti-microtubule herbicides, or nitrous oxide to create homozygous doubled haploid cells.

Methods of chromosome doubling are disclosed in, for example, U.S. Pat. Nos. 5,770,788; 7,135,615, and US Patent Publication No. 2004/0210959 and 2005/0289673; Antoine-Michard, S. et al., Plant Cell, Tissue Organ Cult., Dordrecht, the Netherlands, Kluwer Academic Publishers 48(3):203-207 (1997); Kato, A., Maize Genetics Cooperation Newsletter 1997, 36-37; and Wan, Y. et al., Trends Genetics 77: 889-892 (1989). Wan, Y. et al., Trends Genetics 81: 205-211 (1991), the disclosures of which are incorporated herein by reference. Methods can involve, for example, contacting the haploid cell with nitrous oxide, anti-microtubule herbicides, or colchicine. Optionally, the haploids can be transformed with a heterologous gene of interest, if desired.

Double haploid plants can be further crossed to other plants to generate F1, F2, or subsequent generations of plants with desired traits.

### EXAMPLES

CENH3 is a histone 3 variant that determines, epigenetically, the location of centromeres. Centromeres are the attachment sites for the kinetochore, which is required for the separation of sister chromatids to opposite poles of the cell during mitosis. CENH3 is therefore an essential protein. The protein's structure can be divided into the highly conserved histone fold domain (HFD) and the highly variable N-terminal tail. It is hypothesized that defective (or “weak”) alleles of CENH3 cannot compete with wild-type alleles for kinetochore components (and reloading of centromeric components) during the first few mitotic divisions of embryogenesis. This results in proper segregation of sister chromatids derived from the wild-type parent, but loss of chromosomes derived from the mutant parent.

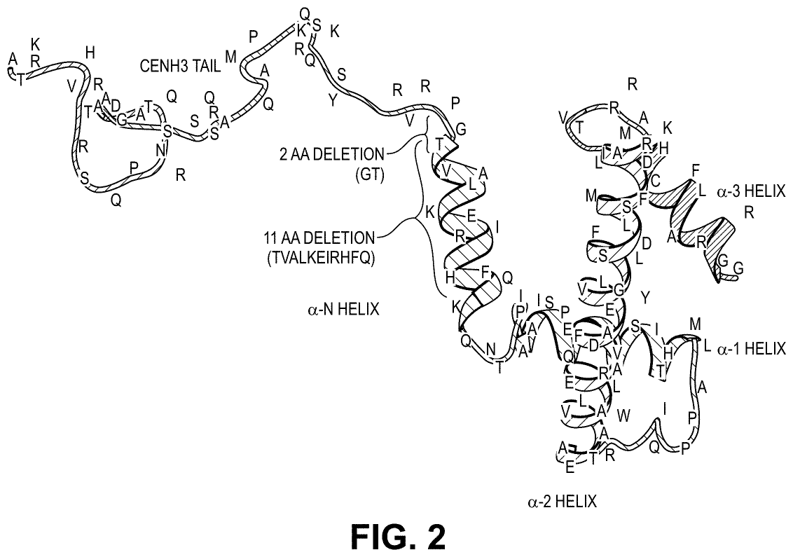

The conservation of the histone fold domain of CENH3 among eukaryotes is illustrated in FIG. 1. We have found surprisingly that the alpha-N helix of the HFD, while conserved in all H3's, is to some extent dispensible for both mitotic and meiotic function of CENH3. Transgenic deletion alleles (FIG. 2) eliminating either the first 2 amino acids, or the 2nd through 11th amino acids of this 15 aa helix, when expressed in Arabidopsis plants that are homozygous null for the endogenous CENH3 allele, result in plants that are strong haploid inducers when crossed by wild-type pollen (approx. 20% of progeny are haploid). We have also shown that expression of a CRISPR/cas carrying either of two guide RNAs that target the junction between the N-terminal domain and the alpha-N helix produce mutations in tomato that result in these same in-frame deletions. Thus, we conclude that plants carrying these (and additional similar) deletion alleles can be generated by CRISPR/cas9, and that the resulting plants will be viable, fertile, and haploid-inducing.

### Details:

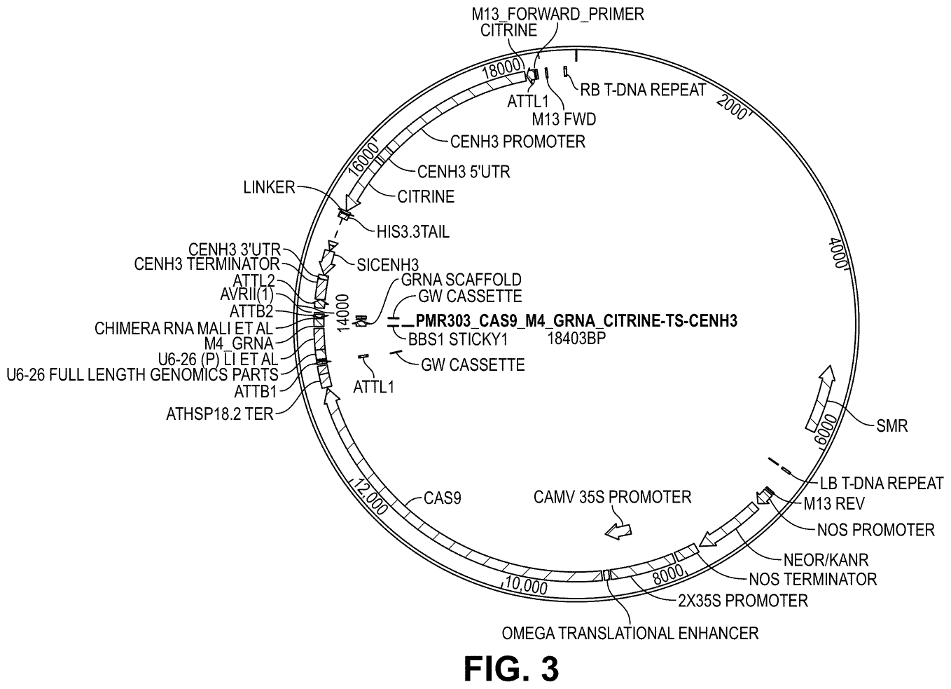

a) Tomatoes were transformed with a variety of T-DNA constructs. The most significant of these, pMR303, carries a CRISPR targeting the region encoding the alpha-N helix of the native CENH3, plus a chimeric CENH3 transgene termed citrine:tailswap (FIG. 3), similar to Chan and Ravi's GFP:tailswap which was a powerful haploid inducer when expressed in CENH3 null Arabidopsis.

Two alleles identified in tomato were:

Δ6-1 (Δ2AA) (SEQ ID NO: 101, Δ6-1) RYRP{GT}VAL (SEQ ID NO: 147) > RYRPVAL (SEQ
ID NO: 148) (bolded and bracketed amino acids in brackets were deleted and
bolded and underlined amino acids were added due to change in the codon).
(SEQ ID NO: 101)
MARTKHLAKRSRTTSAAPSATPSTPSRKSPRSAPATSVQKPKQKKRYRPVALREIRHFQK
TWDLLIPAAPFIRLVREISHFYAPGVTRWQAEALIAIQEAAEDFLVHLFEDAMLCAIHAK
RVTLMKKDFELARRLGGKGQPW*
Δ12 (Δ4AA) (SEQ ID NO: 110, Δ12-3) KKR{YRPGT}VAL (SEQ ID NO: 149) > KKRSVAL
(SEQ ID NO: 150)
(SEQ ID NO: 110)
MARTKHLAKRSRTTSAAPSATPSTPSRKSPRSAPATSVQKPKQKKRSVALREIRHFQKT
VDLLIPAAPFIRLVREISHFYAPGVTRWQAEALIAIQEAAEDFLVHLFEDAMLCAIHAKR
VTLMKKDFELARRLGGKGQPW*

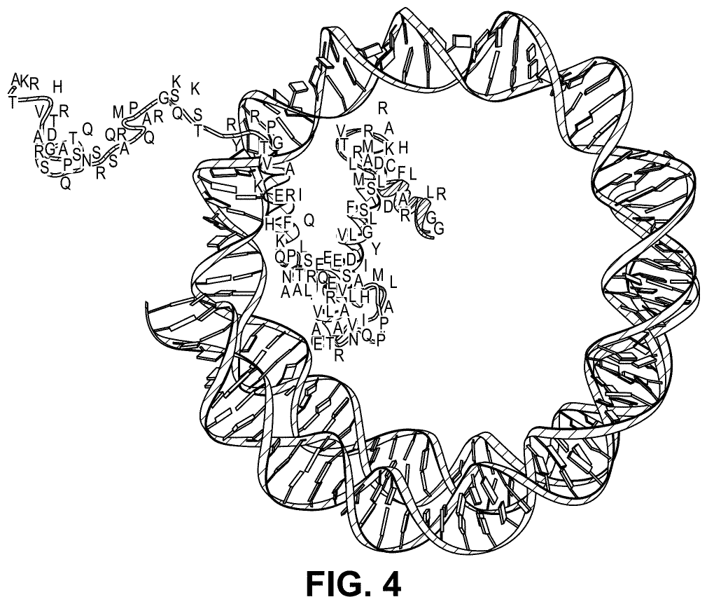

b) Hairy roots transformed with pMR303 often produced homozygous roots carrying in-frame deletions (e.g., Δ3 bp, Δ6 bp, Δ12 bp) and more rarely larger deletions like a 33 bp deletion mutation at CENH3. All of these in-frame deletions are predicted to produce CENH3 proteins with internal deletions within the highly-conserved alpha-N helix (FIGS. 2 and 4). This result suggests that citrine:tailswap can complement a null mutant of CENH3 and/or that these in-frame deletions produce a CENH3 that is mitotically functional.

c) In order to test the functionality of these deletion alleles, Arabidopsis CENH3 alleles with the same deletions were synthesized and transformed into an Arabidopsis CENH3+/−heterozygote. cenh3−/− homozygotes were identified among the T1 transformants. This result indicates that both the 6 bp and the 33 bp deletions express a functional CENH3. The plants are fertile on self-pollination. Outcrossing the deletion mutants by wild-type pollen, in contrast, results in high seed lethality, and production of paternal haploids (assayed as expression of a recessive marker derived from the pollen donor). The two amino acid deletion produces 25% haploids (among surviving seeds), while the eleven amino acid deletion produces 16% haploid progeny (among surviving seeds). Thus CRISPR-induced deletions in the alpha-N helix can result in haploid inducers.

We have demonstrated that, in Arabidopsis, in-frame deletions in the alpha-N helix of CENH3 can induce haploids on outcrossing by wild-type pollen, using transgenic CENH3 variants synthesized in the lab and transformed into CENH3 KO lines.

We have shown that
1) in-frame mutations can routinely be generated by CRISPR mutagenesis using a variety of guides; and
2) a variety of CRISPR-induced in-frame mutations in CENH3 can result in haploid-inducing plants.

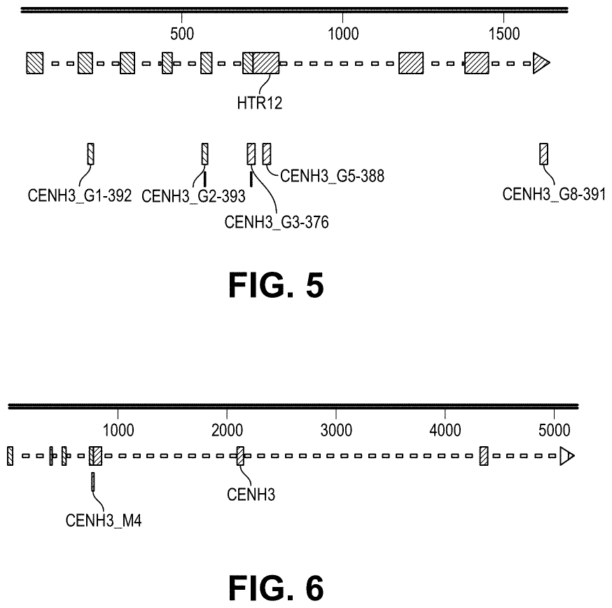

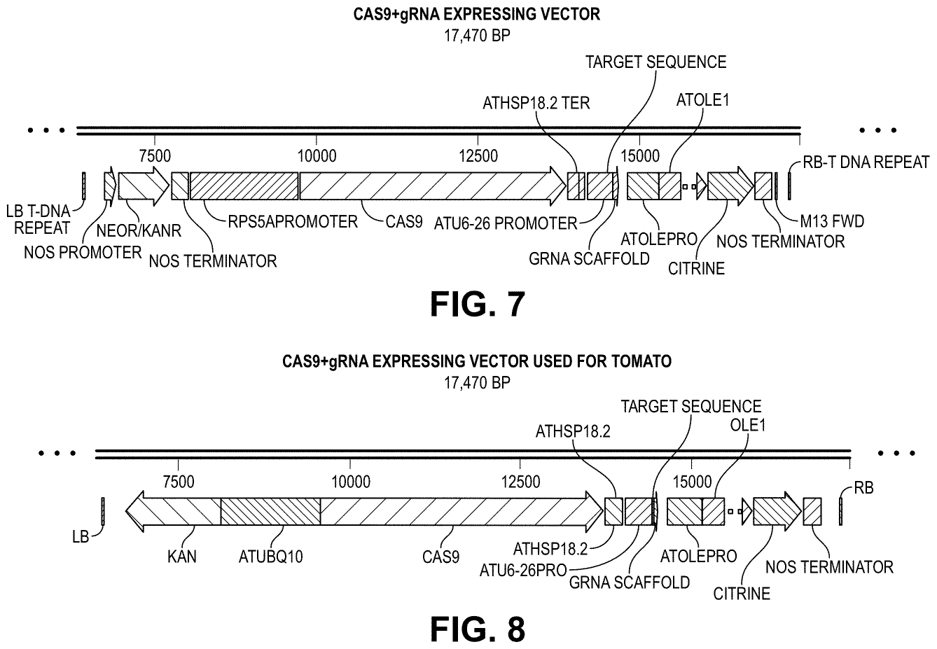

For example we employed 5 guide RNAs distributed across the CENH3 gene of the model plant Arabidopsis to generate in-frame deletions, additions, and amino acid changes. FIG. 5 is a diagram of CENH3. The left portion of the gene is the N-terminal tail and the right side is the histone fold domain, which begins with the alpha-N helix. Our data indicates that some of the resulting mutations result in HI plants. FIG. 7 shows an illustration of a general plasmid used for cloning different gRNAs targeting AtCenH3 and used to transform WT Arabidopsis plants.

The guide RNAs were cloned into a Cas9-expressing vector and the resulting constructs were used to transform WT Col-0 Arabidopsis plants. T1 plants were screened and transgenic plants were genotyped for mutations in CENH3. A list of T2 or T3 mutants obtained as viable homozygotes is provided below. This viability demonstrates that a wide range of changes can be accommodated by CENH3.

From construct CenH3 G1-392 we obtained:

392#2-3 is (+28-4)/(+28-4)(+8AA) GPTTTPT (SEQ ID NO: 151 >
GPTAGPISNLKFTPT (SEQ ID NO: 152) in the N-terminal tail. Bolded and
underlined sequence was added to the wild type CENH3 sequence.
The mutant's protein's sequence is therfore:
(SEQ ID NO: 127)
MARTKHRVTRSQPRNQTDAAGASSSQAAGPTAGPISNLKFTPTRRGGEGGDNTQQTNP
TTSPATGTRRGAKRSRQAMPRGSQKKSYRYRPGTVALKEIRHFQKQTNLLIPAASFIREV
RSITHMLAPPQINRWTAEALVALQEAAEDYLVGLFSDSMLCAIHARRVTLMRKDFELAR
RLGGKGRPW*
392#2-4 is Δ95/Δ95 (Δ3AA) GPTT{TPT}RR (SEQ ID NO: 153) > GPTTRR (SEQ ID
NO: 154) in the N-terminal tail. Bolded and bracketed sequence was deleted
from the wild type CENH3 squence.
The mutant's protein's sequence is therfore:
(SEQ ID NO: 144)
MARTKHRVTRSQPRNQTDAAGASSSQAAGPTTRRGGEGGDNTQQTNPTTSPATGTRRG
AKRSRQAMPRGSQKKSYRYRPGTVALKEIRHFQKQTNLLIPAASFIREVRSITHMLAPPQ
INRWTAEALVALQEAAEDYLVGLFSDSMLCAIHARRVTLMRKDFELARRLGGKGRPW*
392#5-2 is Δ77/Δ77 77 bp of intron causing the 9 bp left to be added (+3AA)
to the N-terminal tail GPTTTPT (SEQ ID NO: 151) > GPTTKLKTPT (SEQ ID NO: 155)
The mutant's protein's sequence is therfore:
(SEQ ID NO: 128)
MARTKHRVTRSQPRNQTDAAGASSSQAAGPTTKLKTPTRRGGEGGDNTQQTNPTTSPA
TGTRRGAKRSRQAMPRGSQKKSYRYPGTVALKEIRHFQKQTNLLIPAASFIREVRSITH
MLAPPQINRWTAEALVALQEAAEDYLVGLFSDSMLCAIHARRVTLMRKDFELARRLGG
KGRPW*
392#9-2 is (+8-)/(+8/-2)(-1+3AA) GPT{T}TPT (SEQ ID NO: 156) > GPTIELTPT
(SEQ ID NO: 157) in the N-terminal tail
The mutant's protein's sequence is therfore:
(SEQ ID NO: 129)
MARTKHRVTRSQPRNQTDAAGASSSQAAGPTIELTPTRRGGEGGDNTQQTNPTTSPAT
GTRRGAKRSRQAMPRGSQKKSYRYRPGTVALKEIRHFQKQTNLLIPAASFIREVRSITHM
LAPPQINRWTAEALVALQEAAEDYLVGLFSDSMLCAIHARRVTLMRKDFELARRLGGK
GRPW*

From construct CenH3 G2-393 we obtained:

393#3-1 is (Δ20/Δ20) has a deletion probably resulting in a splicing defect. This mutation is viable as a homozygote.

393#3-3 is (+3/+3)(+1AA) KRSRQA (SEQ ID NO: 158) > KRSTRQA (SEQ ID NO:
159) in the N-terminal tail
The mutant's protein's sequence is therfore:
(SEQ ID NO: 130)
MARTKHRVTRSQPRNQTDAAGASSSQAAGPTTTPTRRGGEGGDNTQQTNPTTSPATGT
RRGAKRSTRQAMPRGSQKKSYRYRPGTVALKEIRHFQKQTNLLIPAASFIREVRSITHML
APPQINRWTAEALVALQEAAEDYLVGLFSDSMLCAIHARRVTLMRKDFELARRLGGKG
RPW*

From construct CenH3 G1G2-401 we obtained:

401#1-2 is Δ367/Δ367 (Δ37AA)
GPT{TTPTRRGGEGGDNTQQTNPTTSPATGTRRGAKRSRQA}MPR (SEQ ID NO: 160)
> GPTMPR (SEQ ID NO: 161) in the N-terminal tail
The mutant's protein's sequence is therfore:
(SEQ ID NO: 131)
MARTKHRVTRSQPRNQTDAAGASSSQAAGPTMPRGSQKKSYRYRPGTVALKEIRHFQK
QTNLLIPAASFIREVRSITHMLAPPQINRWTAEALVALQEAAEDYLVGLFSDSMLCAIHA
RRVTLMRKDFELARRLGGKGRPW*
401#2-2 is (+15-8)/(+15-8) in G2 that cause splicing change of Δ1AA+7AA
and also Δ9/Δ9 in G2 (Δ3AA)
The mutant's protein's sequence is therfore:
(SEQ ID NO: 132)
MARTKHRVTRSQPRNQTDAAGASSSQAAGPTIVMFLPFSTPTRRGGEGGDNTQQTNPTT
SPATGTRRGAKRSRQAMPRGSQKKSYRYRPGTVALKEIRHFQKQTNLLIPAASFIREVRS
ITHMLAPPQINRWTAEALVALQEAAEDYLVGLFSDSMLCAIHARRVTLMRKDFELARRL
GGKGRPW*
401#3-2 is Δ355/Δ355 (Δ33AA)
GPTT{TPTRRGGEGGDNTQQTNPTTSPATGTRRGAKRS}RQA (SEQ ID NO: 162) >
GPTTRQA (SEQ ID NO: 163)
The mutant's protein's sequence is therfore:
(SEQ ID NO: 133)
MARTKHRVTRSQPRNQTDAAGASSSQAAGPTTRQAMPRGSQKKSYRYRPGTVALKEIR
HFQKQTNLLIPAASFIREVRSITHMLAPPQINRWTAEALVALQEAAEDYLVGLFSDSMLC
AIHARRVTLMRKDFELARRLGGKGRPW*

From construct CenH3 G1G2-355 we got:

58#8 is Δ6/Δ6 (Δ2AA) in G2 AKR{SR}QAM (SEQ ID NO: 164) > AKRQAM (SEQ
ID NO: 165)
The mutant's protein's sequence is therefore:
(SEQ ID NO: 134)
MARTKHRVTRSQPRNQTDAAGASSSQAAGPTTTPTRRGGEGGDNTQQTNPTTSPATGT
RRGAKRQAMPRGSQKKSYRYRPGTVALKEIRHFQKQTNLLIPAASFIREVRSITHMLAPP
QINRWTAEALVALQEAAEDYLVGLFSDSMLCAIHARRVTLMRKDFELARRLGGKGRP
W*
37#1 is Δ408/Δ408 (Δ54)
RNQT{DAAGASSSQAAGPTTTPTRRGGEGGDNTQQTNPTTSPATGTRRGAKRSRQA
MPR)GSQ (SEQ ID NO: 166) > RNQTGSQ (SEQ ID NO: 167)
The mutant's protein's sequence is therefore:
(SEQ ID NO: 135)
MARTKHRVTRSQPRNQTGSQKKSYRYRPGTVALKEIRHFQKQTNLLIPAASFIREVRSIT
HMLAPPQINRWTAEALVALQEAAEDYLVGLFSDSMLCAIHARRVTLMRKDFELARRLG
GKGRPW*

From construct CenH3 G3-376 we got: PGP-26DNA

376#4-5 is (+2-11)/(+2-11)(Δ3AA) KKS{YRYR}PGT (SEQ ID NO: 168) >
KKSMPGT (SEQ ID NO: 169)
The mutant's protein's sequence is therefore:
(SEQ ID NO: 136)
MARTKHRVTRSQPRNQTDAAGASSSQAAGPTTTPTRRGGEGGDNTQQTNPTTSPATGT
RRGAKRSRQAMPRGSQKKSMPGTVALKEIRHFQKQTNLLIPAASFIREVRSITHMLAPPQ
INRWTAEALVALQEAAEDYLVGLFSDSMLCAIHARRVTLMRKDFELARRLGGKGRPW*

From construct CeH3 G5-388 we got:

388#5-1 is (+9-3)/(+9-3)(+2AA) EIRH{FQ}KQTNL (SEQ ID NO: 170) >
EIRHCVIKKQTNL (SEQ ID NO: 171)
The mutant's protein's sequence is therefore:
(SEQ ID NO: 137)
MARTKHRVTRSQPRNQTDAAGASSSQAAGPTTTPTRRGGEGGDNTQQTNPTTSPATGT
RRGAKRSRQAMPRGSQKKSYRYRPGTVALKEIRHCVIKKQTNLLIPAASFIREVRSITHM
LAPPQINRWTAEALVALQEAAEDYLVGLFSDSMLCAIHARRVTLMRKDFELARRLGGK
GRPW*

From construct CenH3 G8-391 we obtained:

391#2-1 is Δ3/Δ3 (Δ1AA) GGK{K}GRPW* (SEQ ID NO: 172) > GGGRPW* (SEQ ID
NO: 173)
The mutant's protein's sequence is therefore:
(SEQ ID NO: 138)
MARTKHRVTRSQPRNQTDAAGASSSQAAGPTTTPTRRGGEGGDNTQQTNPTTSPATGT
RRGAKRSRQAMPRGSQKKSYRYRPGTVALKEIRHFQKQTNLLIPAASFIREVRSITHMLA
PPQINRWTAEALVALQEAAEDYLVGLFSDSMLCAIHARRVTLMRKDFELARRLGGGRP
W*
391#3-1 is Δ9/ (Δ3AA) GG{KGR}PW* (SEQ ID NO: 174) > GGPW* (SEQ ID NO:
175)
The mutant's protein's sequence is therefore:
(SEQ ID NO: 139)
MARTKHRVTRSQPRNQTDAAGASSSQAAGPTTTPTRRGGEGGDNTQQTNPTTSPATGT
RRGAKRSRQAMPRGSQKKSYRYRPGTVALKEIRHFQKQTNLLIPAASFIREVRSITHMLA
PPQINRWTAEALVALQEAAEDYLVGLFSDSMLCAIHARRVTLMRKDFELARRLGGPW*
391#5-3 is (Δ19/Δ19 (remove 6AA + Stop > adding 14 new AA)
RLG{GKGRPW*} (SEEQ ID NO: 176) > RLGDRKLTHYSHLLHCK* (SEQ ID NO: 177)
The mutant's protein's sequence is therefore:
(SEQ ID NO: 140)
MARTKHRVTRSQPRNQTDAAGASSSQAAGPTTTPTRRGGEGGDNTQQTNPTTSPATGT
RRGAKRSRQAMPRGSQKKSYRYRPGTVALKEIRHFQKQTNLLIPAASFIREVRSITHMLA
PPQINRWTAEALVALQEAAEDYLVGLFSDSMLCAIHARRVTLMRKDFELARRLGDRKL
THYSHLLHCK*
391#5-5 is Δ3/Δ3 (Δ1AA) G{G}KGRPW* (SEQ ID NO: 178) > GKGRPW* (SEQ ID
NO: 179)
The mutant's protein's sequence is therefore:
(SEQ ID NO: 141)
MARTKHRVTRSQPRNQTDAAGASSSQAAGPTTTPTRRGGEGGDNTQQTNPTTSPATGT
RRGAKRSRQAMPRGSQKKSYRYRPGTVALKEIRHFQKQTNLLIPAASRIREVRSITHMLA
PPQINRWTAEALVALQEAAEDYLVGLFSDSMLCAIHARRVTLMRKDFELARRLGKGRP
W*
391#6-1 is Δ18/Δ18 (Δ5AA) GG{KGRPW*} (SEQ ID NO: 180) > GG*
The mutant's protein's sequence is therefore:
(SEQ ID NO: 142)
MARTKHRVTRSQPRNQTDAAGASSSQAAGPTTTPTRRGGEGGDNTQQTNPTTSPATGT
RRGAKRSRQAMPRGSQKKSYRYRPGTVALKEIRHFQKQTNLLIPAASFIREVRSITHMLA
PPQINRWTAEALVALQEAAEDYLVGLFSDSMLCAIHARRVTLMRKDFELARRLGG*

Thus we have shown that all guides tested could produce in-frame deletions and additions.

To determine whether these in-frame deletion/addition/substitution lines are haploid inducers, we crossed them by Landsberg erecta glabrous (Ler gl1-1 CE-H3) pollen. Haploid induction was assayed as elimination of maternal (cenh3 mutant derived) chromosomes leading to the production of paternal haploids, which exhibit both of the

recessive erecta and glabrous phenotypes (Kuppu 2015). In this work we scored the frequency of gl (trichomeless) progeny derived from a cross of the mutant CENH3 homozygote by pollen from Ler gl.

The mutant 388#5 (carrying a (+8−2)/(+8−2) bp in-frame addition in the α-N-Helix of the HFD that resulted in a change of two AA and an addition of 2 more AA (EIRH{FQ}KQTNL (SEQ ID NO: 170)>EIRHCVIKKQTNL (SEQ ID NO: 171))), was crossed by the tester pollen (Ler gl1-1 CENH3). Among the offspring 8.6% (7 out of 81) were trichomeless, consistent with loss of the dominant maternal marker gl1 (the marker er was not tested in any of these experiments).

The mutant 376#4 (carrying a Δ9/Δ9 bp in-frame deletion in the Tail-HFD junction, resulting in a change of 1 AA and deletion of 3 more AA (KKS{YRYR} (SEQ ID NO: 168)>KKSMPGT (SEQ ID NO: 169)), when crossed with the tester pollen produced 3% (4 out of 133) trichomeless offspring.

The mutant 58#8 (carring a Δ6/Δ6 bp in-frame deletion in the tail domain resulting in a deletion of 2 AA (AKR{SR}QAM (SEQ ID NO: 164)>AKRQAM (SEQ ID NO: 165)), when crossed with the tester pollen produced 0.5% (2 out of 376) trichomeless offspring.

The mutant 392#2-3 (carrying a (+28−4)/(+28−4) bp in-frame addition in the tail domain that results in an addition of 8

AA(GPTTFPT (SEQ ID NO: 151) > GPTAGPISNLKFTPT
(SEQ ID NO: 152)),

when crossed with the tester pollen, produced 0.3%(1 out of 328) trichomeless offspring.

In addition, in order to test this method in other crops we designed a construct with a gRNA to target the α-N-Helix of the HFD in tomato. See FIG. 6, depicting the tomato CENH3 gene with exons indicated. Again the left portion is the tail and the right portion is the histone fold domain. FIG. 8 shows an illustration of a general plasmid used for cloning gRNA targeting S1CenH3 and used to transform WT tomato plants.

From transformation events we identified 3 plants carrying in-frame deletions of either

Δ6 bp (RYRP{GT}VAL (SEQ ID NO: 147) > RYRPVAL (SEQ ID NO: 148)) or A9 bp
RY{RPG}TVAL (SEQ ID NO: 181) > RYTVAL (SEQ ID NO: 182).
A6-1 (A2AA) (this is the same allele as SEQ ID NO: 101, A6-1) RYRP{GT}VAL
(SEQ ID NO: 147) > RYRPVAL (SEQ ID NO: 148)
(SEQ ID NO: 101)
MARTKHLAKRSRTTSAAPSATPSTPSRKSPRSAPATSVQKPKQKKRYRPVALREIRHFQK
TWDLLIPAAPFIRLVREISHFYAPGVTRWQAEALIAIQEAAEDFLVHLFEDAMLCAIHAK
RVTLMKKDFELARRLGGKGQPW*
A9-1
(SEQ ID NO: 143)
MARTKHLAKRSRTTSAAPSATPSTPSRKSPRSAPATSVQKPKQKKRYTVALREIRHFQKT
WDLKIPAAPFIRLVREISHFYAPGVTRWQAEALIAIQEAAEDFLVHLFEDAMLCAIHAKR
VTLMKKDFELARRLGGKGQPW*

We have also derived a tomato line homozygous for allele Δ6-1 without the citrine:tailswap and found that it is viable and fertile. This result suggests that these in-frame deletions can produce a CENH3 that is both mitotically and meioticaly functional.

In summary, we found, that all of our guides could produce mutations that result in amino acid in-frame indels at the target site. Our results indicate that the in-frame indels in the HFD has a stronger effect on the ability to induce haploids than indels in the N-terminal tail, but indels in either domain are capable of generating a haploid-inducing allele.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of creating a haploid inducing plant, the method comprising,
editing a CENH3 gene of a plant such that the CENH3 gene encodes a CENH3 polypeptide with a two or more contiguous amino acid deletion relative to wild-type CENH3, wherein said haploid inducing plant, when crossed with a second plant, results in haploid progeny.

2. The method of claim 1, wherein the CENH3 polypeptide has an eleven amino acid deletion relative to wild-type CENH3.

3. The method of claim 1, wherein the CENH3 polypeptide has a 2-15 (e.g., 2-12) contiguous amino acid deletion relative to wild-type CENH3.

4. The method of claim 1, wherein the deletion is in a alpha-N helix domain of the CENH3 polypeptide.

5. The method of claim 1, wherein the CENH3 polypeptide comprises a sequence at least 90% identical to SEQ ID NO:1-50 or 101-126.

6. The method of claim 1, wherein the CENH3 polypeptide comprises any of SEQ ID NO: 101, 110, 116-117, or 126-144.

7. The method of claim 1, wherein the plant is a tomato or potato plant.

8. The method of claim 1, wherein the editing occurs in situ in the plant.

9. The method of claim 1, wherein the editing comprises introducing into the plant a Cas protein or Cpf1 protein and a guide RNA targeting a CENH3-coding sequence, thereby inducing the two or more contiguous amino acid deletion.

10. A method of creating a haploid inducing plant, the method comprising,
editing a CENH3 gene of a plant such that the CENH3 gene encodes a CENH3 polypeptide with a two or more contiguous amino acid insertion relative to wild-type CENH3, wherein said haploid inducing plant, when crossed with a second plant, results in haploid progeny.

11. The method of claim 10, wherein the CENH3 polypeptide has a 2-15 contiguous amino acid insertion relative to wild-type CENH3.

12. The method of claim 10, wherein the insertion is in an alpha-N helix domain of the CENH3 polypeptide.

13. The method of claim 10, wherein the CENH3 polypeptide comprises a sequence at least 90% identical to SEQ ID NO:1-50 or 101-126.

14. The method of claim 10, wherein the CENH3 polypeptide comprises any of SEQ ID NO: 101, 110, 116-117, or 126-144.

15. The method of claim 10, wherein the plant is a tomato or potato plant.

16. The method of claim 10, wherein the editing occurs in situ in the plant.

17. The method of claim 10, wherein the editing comprises introducing into the plant a Cas protein or Cpf1 protein and a guide RNA targeting a CENH3-coding sequence, thereby inducing the two or more contiguous amino acid insertion.

18. A haploid-inducing plant expressing a mutant CENH3 polypeptide encoded by a CENH3 coding sequence, wherein the CENH3 coding sequence comprises an in-frame deletion or insertion of 6 or more contiguous nucleotides, relative to wildtype CENH3.

19. The haploid-inducing plant of claim 18, wherein the in-frame deletion comprises 6-42 contiguous nucleotides of the wildtype CENH3 gene.

20-24. (canceled)

21. A method of making progeny with reduced chromosome content, the method comprising
crossing the plant of claim 18 to a plant having a ploidy; and
selecting progeny from the cross that have half the ploidy.

22-28. (canceled)


SEQUENCE LISTING (195 sequences)

SEQ ID NO: 1 - protein, 136 aa, Arabidopsis thaliana
MARTKQSARKSHGGKAPTKQLATKAARKSAPTTGGVKKPHRFRPGTVALREIRKYQKSTELLNRKLPFQRLVREIAQDFKTDLRFQSHAVLALQEAAEAYLVGLFEDTNLCAIHAKRVTIMPKDVQLARRIRAERA

SEQ ID NO: 2 - protein, 136 aa, Homo sapiens
MARTKQTARKSTGGKAPRKQLATKAARKSAPSTGGVKKPHRYRPGTVALREIRRYQKSTELLIRKLPFQRLVREIAQDFKTDLRFQSAAIGALQEASEAYLVGLFEDTNLCAIHAKRVTIMPKDIQLARRIRGERA

SEQ ID NO: 3 - protein, 125 aa, Physcomitrella patens
MARRKTTPVHGNHRASTSSVGGAAVRPRKPHRWRPGTKALQEIRHYQKTCDLLIPRLPFARYVKEITMMYASDVSRWTAEALTALQEATEDYMCHLFEDTNLCAIHAKRVTIMPKDLQLARRLRG

SEQ ID NO: 4 - protein, 164 aa, Pinus taeda
MVRRKTVPPRKKSGSGNAASTSGVGVSTPGSAGERGERRGSARLASTPGSDASPSAPSGRKPHRFRPGTVALREIKRYQKSFELLIPSLPFARIVRELTMYYSQVVSRWAAEALVALQEAAEDYIVHLFEDTNLCAIHAKRVTIMPRDLRLARRLRGGGLDRPW

SEQ ID NO: 5 - protein, 177 aa, Boechera holboelli
MARTKHLATRSRPRNQTDATASSSQAAGPSTNPTTRGSEGEDAAQETTPTTSPATGRKKGAKRARYARPQGSQKKPYRYKPGTVALREIRYFQKSINLLIPAASFIRQVRSITHALAPPQITRWTAEALVALQEAAEDYLVGLFSDSMLCAIHARRVTLMRKDFELARRLGGKGRPW

SEQ ID NO: 6 - protein, 177 aa, Boechera stricta
MARTKHLATRSRPRNWTDATASSSQAAGPTTNPTTRGSEGEDAAQEPTPTTSPATGRKKGAKRARYARPQGSQKKPYRYKPGTVALREIRHFQKSINLLIPAASFIRQVRSITHALAPPQITRWTAEALVALQEAAEDYLVGLFSDSMLCAIHARRITLMRKDFELARRLGGKGRPW

SEQ ID NO: 7 - protein, 180 aa, Lepidium virginicum
MARTKRYASRPQRPRNQTDVTVPSSPAAGPSTNPTRRDSEGEGGDDAQQTVPTTSPASISKKASKKNRKATPQSSKKKTYRYKPGTVALREIRHFQKSTHLLIPAAAFIREVRCITQAVAPPQISRWTAEALVALQEAAEDYVVGLLSDSMLCAIHARRVTLMRKDFELARRLGGKGRPW

SEQ ID NO: 8 - protein, 172 aa, Cardaminopsis flexuosa
MARTKHFPNRTRPRNQTDATTPAAGPSTRTTRANQGEETQQTNPTTSPATSKKKGAKRTRRDMPQGSQKKPYRYKPGTVALREIRHFQKSTNLLIPAASFIRQVRSITQMYAPPQINRWTAEALVALQEAAEDYLVGLFSDSMLCAIHARRVTLMRKDFELARRLGGKGRPW

SEQ ID NO: 9 - protein, 139 aa, Hordeum vulgare
MARTKKTVAAKEKRPPCSKSEPQSQPKKKEKRAYRFRPGTVALREIRKYRKSTNMLIPFAPFVRLVRDIADNLTPLSNKKESKPTPWTPLALLSLQESAEYHLVDLFGKANLCAIHSHRVTIMLKDMQLARRIGTRSLW

SEQ ID NO: 10 - protein, 178 aa, Arabidopsis thaliana
MARTKHRVTRSQPRNQTDAAGASSSQAAGPTTTPTRRGGEGGDNTQQTNPTTSPATGTRRGAKRSRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 11 - protein, 152 aa, Populus trichocarpa
MARTKHPVARKRARSPKRSDASPSTPRTPTSSRTRPQANGQQGSSTQRQRKKHRFRSGTVALREIRQYQKTWRPLIPAASFIRCVRMITQEFSREVNRWTAEALVAIQEAAEDFLVHLFEDGMLCAIHAKRVTLMKKDFELARRLGGKGRPW

SEQ ID NO: 12 - protein, 166 aa, Triticum aestivum
MARTKHPAVRKTKALPKKQLGTRPSAGTPRRQETDGAGTSATPRRAGRAAAPGAAEGATGQPKQRKPHRFRPGTVALREIRKYQKSVDFLIPFAPFVRLIKEVTDFFCPEISRWTPQALVAIQEAAEYHLVDVFERANHCAIHAKRVTVMQKDIQLARRIGGRRLW

SEQ ID NO: 13 - protein, 170 aa, Oryza sativa
MARTKHPAVRKSKAEPKKKLQFERSPRPSKAQRAGGGTGTSATTRSAAGTSASGTPRQQTKQRKPHRFRPGTVALREIRKFQKTTELLIPFAPFSRLVREITDFYSKDVSRWTLEALLALQEAAEYHLVDIFEVSNLCAIHAKRVTIMQKDMQLARRIGGRRPWNLNSLR

SEQ ID NO: 14 - protein, 167 aa, Luzula nivea
MARTKHFPQCSRHPKKQRTAAGEAGSSVIAKQNAPAKTGNASSITNSTPARSLKKNKASKRGEKTQAKQRKMYRYRPGTVALREIRKLQKTTDLLVPKASFARLVKEITFQSSKEVNRWQAEALIALQEASECFLVNLLESANMLAIHARRVTIMKKDIQLARRIGA

SEQ ID NO: 15 - protein, 176 aa, Arabidopsis arenosa
MARTKHFATRTGSGNRTDANASSSQAAGPTTTPTTRGTEGGDNTQQTNPTTSPATGGRRPRRARQAMPRGSQKKPYRYKPGTVALREIRHFQKQTNLLIPAASFIRQVRSITHALAPPQINRWTAEALVALQEAAEDYLIGLFSDSMLCAIHARRVTLMRKDFELARRLGGKGRPW

SEQ ID NO: 16 - protein, 157 aa, Zea mays
MARTKHQAVRKTAEKPKKKLQFERSGGASTSATPERAAGTGGRAASGGDSVKKTKPRHRWRPGTVALREIRKYQKSTEPLIPFAPFVRVVRELTNFVTNGKVERYTAEALLALQEAAEFHLIELFEMANLCAIHAKRVTIMQKDIQLARRIGGRRWA

SEQ ID NO: 17 - protein, 154 aa, Sorghum bicolor
MARTKHQAVRKLPQKPKKKLQFERAGGASTSATPRRNAGTGGGAAARGEDLFKKHRWRAGTVALREIRKYQKSTEPLIPFAPFVRVVKELTAFITDWRIGRYTPEALLALQEAAEFHLIELFEVANLCAIHAKRVTVMQKDIQLARRIGGRRWS

SEQ ID NO: 18 - protein, 150 aa, Cichorium intybus
MARTKQPAKRSWGNRKSSQSRASTSTSTSTPRKSPRKDPGRTGERRQQKPHRFKPGAQALREIRRLQKTVNLLIPAAPFIRTVKEISNYIAPEVTRWQAEAIQALQEAAEDYLVQLFEDSMLCSIHAKRVTLMKKDWELARRLTKKGQPW

SEQ ID NO: 19 - protein, 153 aa, Cycas rumphii
MARKKASTPRKKTGTAASTSAVESPPSGVNQTARARRSVGGVAPGAPRTPQASTNVGTPRRPHRFRPGTVALREIRRYQKSFELLIPALPFARNVRELTLHHSREVHRWTAEALVALQEAAEDYIVHLFEDTNLCAIHAKRVTIMPKDMHLAR

SEQ ID NO: 20 - protein, 154 aa, Allium cepa
MARTKQMAHKKLRRKLNVDEAGPSTPVTRSTEVNPKSSRPTPITEDRGTGARKKHRFRPGTVALREIRKYQKTAELLIPAAPFIRLVREITNLYSKEVTRWTPEALLAIQEAAEFFIINLLEEANLCAIHAKRVTLMQKDIQLARRIGGARHFS

SEQ ID NO: 21 - protein, 199 aa, Malus domestica
MARIKHTAHKKSVARKSSTPKEAAAGTGGTSAASPAKQPEPSAPWRRSERSSQRTSESQEQQEPETNAQATPQSKKQKQSERNPQTPQSKKQKPSERNPPPTQKKKWRYRPGTVALREIRYYQKTWNLIIPAAPFIRTVREISINMSKDPVRWTPEALQAIQEAAEDFLVRLFEDSMLCAIHARRVTLMKKDLELARRI

SEQ ID NO: 22 - protein, 150 aa, Lactuta sativa
MARTKQPAKRSWGKRQSAGASTSTSTSTPRKSPRKDPGSSGTGQRQKQKPHRFKPGTQALREIRRLQKTVNLLIPAAPFIRTVKEISNYIAPEVTRWQAEALQALQEAAEDYIVQLFEDSMLCSIHAKRVTLMKKDMELARRLTKKGQPW

SEQ ID NO: 23 - protein, 145 aa, Carthamus tinctorius
MARTKQPAKRSSGKRDARPSTSTPTPRPSARKNPESSGAGDGQRRHRYRPGTQALREIRRLQKTVNLLIPAAPFIRTVKEISNYIAPEVTRWQAEALQALQEAAEDYLIQLFEDSMLCAIHAKRVTLMKKDWELARRLGKKGQPW

SEQ ID NO: 24 - protein, 145 aa, Helianthus exilis; identical to SEQ ID NO: 23

SEQ ID NO: 25 - protein, 150 aa, Gossypium hirsutum
MSRTKHTAAKKPRRKPSAAAAASPATASPHTRSVTAKKTGGPATPTPGKSKRPHRFRAGTRALQEIRKYQKTSNLLVPAASFIREVRAISYRFAPDINRWQAEALVAIQEAEDYLIQLFGDAMLCAIHAKRVTLMKKDIQLARRLGGMGQ

SEQ ID NO: 26 - protein, 155 aa, Glycine max
MARVKHTPASRKSAKKQAPRASTSTQPPPQSQSPATRERRRAQQVEPQQEPEAQGRKKRRNRSGTVALREIRHFQRSCELLIPAAPFIRCVKQITNQFSTEVSRWTPEAVVALQEAAEEYLVHLFEDGMLCAIHARRITLMKKDIELARRLGGIG

SEQ ID NO: 27 - protein, 153 aa, Cucumis melo
MARARHPVQRKSNRTSSGSGAALSPPAVPSTPLNGRTQNVRKAQSPPSRTKKKIRFRPGTVALREIRNLQKSWNLLIPASCFIRAVKEVSNQLAPQITRWQAEALVALQEAAEDFLVHLFEDTMLCAIHAKRVTIMKKDFELARRLGGKGRPW

SEQ ID NO: 28 - protein, 147 aa, Solanum chacoense
MARTKHLAKRSRTKPSVAAGPSATPSTPTRKSPRSAPATSVPKPKQKKRYRPGSVALREIRHFQKTWNLVIPAAPFIRLVREISHFFAPGVTRWQAEALIAIQEAAEDFLVHLFEDAMLCAIHAKRVTLMKKDFELARRLGGKGQPW

SEQ ID NO: 29 - protein, 144 aa, Solanum lycopersicum
MARTKHLAKRSRTTSAAPSATPSTPSRKSPRSAPATSVQKPKQKKRYRPGTVALREIRHFQKTWDLLIPAAPFIRLVREISHFYAPGVTRWQAEALIAIQEAAEDFLVHLFEDAMLCAIHAKRVTLMKKDFELARRLGGKGQPW

SEQ ID NO: 30 - protein, 156 aa, Nicotiana tabacum
MARTKHLALRKQSRPPSRPTATRSAAAAASSAPQSTPTRTSQRTAPSTPGRTQKKKTRYRPGTVALREIRRFQKTWDLLIPAAPFIRLVKEISHFFAPEVTRWQAEALIALQEAAEDFLVHLFDDSMLCAIHAKRVTLMKKDFELARRLGGKARPW

SEQ ID NO: 31 - protein, 120 aa, Nicotiana tabacum
MARTKHLALRKQSRPPSRPTATRSAAAAASSSAPQSTPTRTSQRTAPSTPGRTQKKKTRYRPGTVALREIRRFQKTWNLLIPAAPFIRLVKEISYFFAPEVTRWQAEALIALQEAAEDFL

SEQ ID NO: 32 - protein, 156 aa, Nicotiana tomentosiformis; identical to SEQ ID NO: 30

SEQ ID NO: 33 - protein, 158 aa, Vitis vinifera
MTRTKHLARKSRNRRRQFAATPASPASAGPSSAPPRRPTRTATDASPSTAGSQGQRKPFRYRPGTVALREIRRFQKTTHLLIPAAPFIRTVREISYFFAPEISRWTAEALVALQEAAEDYLVHLFEDAMLCAIHAKRVTLMKKDWELARRIGGKGQPW

SEQ ID NO: 34 - protein, 157 aa, Nicotiana sylvestris
MARTKHLALRKQSRPPSRPTATRSAAAAASSSAPQSTPTRTSQRTAPSTPGRTQKKKTRYRPGTVALREIRRFQKTWNLLIPAAPFIRLVKEISYFFAPEVTRWQAEALIALQEAAEDFLVHLFDDSMLCAIHAKRVTLMKKDFELARRLGGKARPW

SEQ ID NO: 35 - protein, 177 aa, Crucihimalaya himalaica
MARTKHFATRSRPRNQTDATASASQATGPSTNPTTRGSEGEDAARGTNPTTSPATGRKKGVKRARHAMPQGSQKKPYRYKAGTVALREIRHFQKNTNLLIPAASFIRQVKSITYAVAPPQITRWTAEALVALQEAAEDYLVGLFSDSMLCAIHARRVTLMRKDFELARRLGGKGRPW

SEQ ID NO: 36 - protein, 176 aa, Arabidopsis lyrata
MARTKHFATKSRSGNRTDANASSSQAAGPTTTPTTRGTEGGDNTQQTNPTTSPATGGRRPRRARQAMPRVSQNKPYRYKPGTVALREIRHFQKQTNLLIPAASFIRQVRSITHALAPPQINRWTAEALVALQEAAEDYLVGLFSDSMLCAIHARRVTLMRKDFELARRLGGKGRPW

SEQ ID NO: 37 - protein, 170 aa, Capsella bursapastoris
MARTKHFATRSGPRTPAVASSSQAAVPSSSPATRGRVGVDAAAQQPTPATSPATAKKKGAKRARFGRPQGSQKKKPYRYRPGTVALREIRHYQKGTSLLIPAAAFIRQVRSITNAVAPREVNRWTAEALVALQEAAEDFLVGLFSDSMLCAIHARRVTLMRKDFDLARRL

SEQ ID NO: 38 - protein, 178 aa, Raphanus sativus
MARTKHFASRARDRNQPNAAAAAAGPSATPTRRGSSQGEEAQQTTPTTTSPATTASGRKKGTKRTTQAMPKSSKKKTFRYKPGTVALREIRHFQKSTKLLIPSAPFIREVRSITHNLAAAYVTRWTAEALIALQEAAEDFLVGLFSDAMLCAIHAKRVTLMRKDFELARRLGGKGRPF

SEQ ID NO: 39 - protein, 164 aa, Eruca sativa
MARTKHFASRARDRNRNNATASSSAAAAAAGPSATPTRRGSRQGGGGGGGVEAQQGSNKKKKSFRYKPGTVALREIRHFQKTTKLLIPAATFIRLVRSITLDRAKPQVTRWTAEALVALQEAAEDYLVGLFSDSMLCAIHAKRVTLMRKDFELARRLGGKGRPW

SEQ ID NO: 40 - protein, 173 aa, Olimarabidopsis pumila
MARTKHNAIRSRDRTGATASSSQAAGPSTNPTAGGSEDAAQQTTPTTSPATGSKKRAKRARQAMPRGSQKKPYRYKPGTVALREIRHFQKTTSLLLPAAPFIRQVRSISSALAPREITRWTAEALVALQEAAEDYLVGLFSDSMLCAIHAKRVTLMRKDFELARRLGGKGRPW

SEQ ID NO: 41 - protein, 174 aa, Olimarabidopsis pumila
MTRTKHTVIKSSRPLDRTDASSSQAAGPSTNPTAGSSGDAAQQTTPTTSPATGSTKRAKRARQAMPRGSQKKPYRYKPGTVALREIRHFQKTTSFLIPAAPFIRQVRSISSALAPTQITRWTAEALVALQEAAEDYLVGLFSDSMLCAIHAKRVTLMRKDFELARRLGGKGRPW

SEQ ID NO: 42 - protein, 174 aa, Turritis glabra
MARTKHFATRSRPRNQTDSSSQAAGPSTNPTTGGSEGGDAAQQTTPTTSPATGRKKRAKRAKQAMPQGSQKKPYRYKPGTIALREIRYFQKNTNLLIPAASFIREVRSITHALAPPQISRWTAEALVALQEAAEDYLVGLFSDSMLCAIHARRVTLMRKDFELARRIGGKGRPW

SEQ ID NO: 43 - protein, 176 aa, Arabidopsis halleri
MARTKHFAIKSRSGNRTDANASSSQAAGPTTTPTTRGTEGGDNTQQTNPTTSPATGGRRPRRARQAMPRGSQKKPYRYKPGTVALREIRHFQKQTNLLIPAASFIRQVRSITHALAPPQINRWTAEALVALQEAAEDYLVGLFSDSMLCAIHARRVTLMRKDFELTRRLGGKGRPW

SEQ ID NO: 44 - protein, 176 aa, Arabidopsis halleri
MARTKHFVTRKGSGNRTDFDANASSSQAAGPTKTPTTRGTEGGDNTQQTTSPATGGRRGPRRARQAMPRGSQKKPYRYKPGTVALREIRHFQKQTNLLIPAASFIRQVRSITHALAPPQINRWTAEALVALQEAAEDYLVGLFSDSMLCAIHARRVTLMRKDFELARRLGGKGRPW

SEQ ID NO: 45 - protein, 175 aa, Arabidopsis lyrata
MARTKHFATRTGSGNRTDANASSSSQAAGPTKTPTTRGTEGGDNTQQTTSPATGGRRGPRRARQAMPRGSQKKPYRYKPGTVALREIRHFQKQTNLLIPAASFIRQARSITHALAPPQINRWTAEALVALQEAAEDYLVGLFSDSMLCAIHARRVTLMRKDFELARRLGGKGRPW

SEQ ID NO: 46 - protein, 172 aa, Arabidopsis lyrata
MARTKHFATKSRTDANASSSQAAGPTTTPTTRGTEGGDNTQQTNPTTSPATGGRRPRRARQAMPRGSQKKPYRYKPGTVALREIRHFQKQTNLLIPAASFIRQVRSITHALAPPQINRWTAEALVALQEAAEDYLVGLFSDSMLCAIHARRVTLMRKDFELARRLGGKGRPW

SEQ ID NO: 47 - protein, 163 aa, Saccharum officinalis
MARTKHQAVRRPTQKPKKKLQFERAGGASTSATPERNAGTGGGAAARVTRGRVEKKHRWRVGTVALREIRKYQKSTEPLIPFAPFVRVVKELTGFITDWRIGRYTPEALLALQEAAEFHLIELFQVANLCAIHAKRVTVMQKDIQLARRIGGKRWAYPFFLPY

SEQ ID NO: 48 - protein, 181 aa, Brassica napa
MARTKHFASRARDRNPTNATASSSAAAAAGPSATPTRRGGSQGGEAQQTTPPATTTAGRKKGGTKRTKQAMPKSSNKKKTFRYKPGTVALREIRHFQKTTKLLIPAASFIREVRSVTQIFAPPDVTRWTAEALMAIQEAAEDFLVGLFSDAMLCAIHARRVTLMRKDFELARRLGGKGRPL

SEQ ID NO: 49 - protein, 180 aa, Lepidium oleraceum
MARTKRFASRPQRPRNQTDTTVPSSPAAGPSTNPTRRDSEGEGGDDAQQTVPTTSPATTSKKVSKRTGKVMPQSSKKKTYRYKPGTVALREIRHFQKSTHFLIPAAAFIREVRCITQAVAPPQISRWTAEALVALQEAAEDYVVGLLSDSMLCAIHARRVTLMRKDFELARRLGGKGRPW

SEQ ID NO: 50 - protein, 182 aa, Brassica rapa
MARTKHFASRARDRNPTNATASSSAAAAAGPSATPTRRGGSQGGEAQQTATPPATTTAGRKKGGTKRTKQAMPKSSNKKKTFRYKPGTVALREIRHFQKTTKLLIPAASFIREVRSVTQIFAPPDVTRWTAEALMAIQEAAEDFLVGLFSDAMLCAIHARRVTLMRKDFELARRLGGKGRPL

SEQ ID NO: 51-54 - skipped (4 entries)

SEQ ID NO: 55 - protein, 102 aa, Artificial Sequence
  Description of Artificial Sequence: Synthetic polypeptide
  Hordeum vulgare barley CENH3 histone domain
PGTVALREIRKYRKSTNMLIPFAPFVRLVRDIADNLTPLSNKKESKPTPWTPLALLSLQESAEYHLVDLFGKANLCAIHSHRVTIMLKDMQLARRIGTRSLW

SEQ ID NO: 56 - protein, 97 aa, Artificial Sequence
  Description of Artificial Sequence: Synthetic polypeptide
  Arabidopsis thaliana thale cress CENH3 histone domain
PGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 57 - protein, 96 aa, Artificial Sequence
  Description of Artificial Sequence: Synthetic polypeptide
  Populus trichocarpa black cottonwood CENH3 histone domain
SGTVALREIRQYQKTWRPLIPAASFIRCVRMITQEFSREVNRWTAEALVAIQEAAEDFLVHLFEDGMLCAIHAKRVTLMKKDFELARRLGGKGRPW

SEQ ID NO: 58 - protein, 95 aa, Artificial Sequence
  Description of Artificial Sequence: Synthetic polypeptide
  Triticum aestivum wheat CENH3 histone domain
PGTVALREIRKYQKSVDFLIPFAPFVRLIKEVTDFFCPEISRWTPQALVAIQEAAEYHLVDVFERANHCAIHAKRVTVMQKDIQLARRIGGRRLW

SEQ ID NO: 59 - protein, 101 aa, Artificial Sequence
  Description of Artificial Sequence: Synthetic polypeptide
  Oryza sativa rice CENH3 histone domain
PGTVALREIRKFQKTTELLIPFAPFSRLVREITDFYSKDVSRWTLEALLALQEAAEYHLVDIFEVSNLCAIHAKRVTIMQKDMQLARRIGGRRPWNLNSLR

SEQ ID NO: 60 - protein, 91 aa, Artificial Sequence
  Description of Artificial Sequence: Synthetic polypeptide
  Luzula nivea snowy woodrush CENH3 histone domain
PGTVALREIRKLQKTTDLLVPKASFARLVKEITFQSSKEVNRWQAEALIALQEASECFLVNLLESANMLAIHARRVTIMKKDIQLARRIGA

SEQ ID NO: 61 - protein, 97 aa, Artificial Sequence
  Description of Artificial Sequence: Synthetic polypeptide
  Arabidopsis arenosa sand rockcress CENH3 histone domain
PGTVALREIRHFQKQTNLLIPAASFIRQVRSITHALAPPQINRWTAEALVALQEAAEDYLIGLFSDSMLCAIHARRVTLMRKDFELARRLGGKGRPW

SEQ ID NO: 62 - protein, 96 aa, Artificial Sequence
  Description of Artificial Sequence: Synthetic polypeptide
  Zea mays corn CENH3 histone domain
PGTVALREIRKYQKSTEPLIPFAPFVRVVRELTNFVTNGKVERYTAEALLALQEAAEFHLIELFEMANLCAIHAKRVTIMQKDIQLARRIGGRRWA

SEQ ID NO: 63 - protein, 96 aa, Artificial Sequence
  Description of Artificial Sequence: Synthetic polypeptide
  Sorghum bicolor sorghum CENH3 histone domain
AGTVALREIRKYQKSTEPLIPFAPFVRVVKELTAFITDWRIGRYTPEALLALQEAAEFHLIELFEVANLCAIHAKRVTVMQKDIQLARRIGGRRWS

SEQ ID NO: 64 - protein, 96 aa, Artificial Sequence
  Description of Artificial Sequence: Synthetic polypeptide
  Cichorium intybus chicory CENH3 histone domain
PGAQALREIRRLQKTVNLLIPAAPFIRTVKEISNYIAPEVTRWQAEAIQALQEAAEDYLVQLFEDSMLCSIHAKRVTLMKKDWELARRLTKKGQPW

SEQ ID NO: 65 - protein, 87 aa, Artificial Sequence
  Description of Artificial Sequence: Synthetic polypeptide
  Cycas rumphii queen sago CENH3 histone domain
PGTVALREIRRYQKSFELLIPALPFARNVRELTLHHSREVHRWTAEALVALQEAAEDYIVHLFEDTNLCAIHAKRVTIMPKDMHLAR

SEQ ID NO: 66 - protein, 96 aa, Artificial Sequence
  Description of Artificial Sequence: Synthetic polypeptide
  Allium cepa onion CENH3 histone domain
PGTVALREIRKYQKTAELLIPAAPFIRLVREITNLYSKEVTRWTPEALLAIQEAAEFFIINLLEEANLCAIHAKRVTLMQKDIQLARRIGGARHFS

SEQ ID NO: 67 - protein, 89 aa, Artificial Sequence
  Description of Artificial Sequence: Synthetic polypeptide
  Malus domestica apple CENH3 histone domain
PGTVALREIRYYQKTWNLIIPAAPFIRTVREISINMSKDPVRWTPEALQAIQEAAEDFLVRLFEDSMLCAIHARRVTLMKKDLELARRI

SEQ ID NO: 68 - protein, 96 aa, Artificial Sequence
  Description of Artificial Sequence: Synthetic polypeptide
  Lactuca sativa lettuce CENH3 histone domain
PGTQALREIRRLQKTVNLLIPAAPFIRTVKEISNYIAPEVTRWQAEALQALQEAAEDYIVQLFEDSMLCSIHAKRVTLMKKDMELARRLTKKGQPW

SEQ ID NO: 69 - protein, 96 aa, Artificial Sequence
  Description of Artificial Sequence: Synthetic polypeptide
  Carthamus tinctorius safflower CENH3 histone domain
PGTQALREIRRLQKTVNLLIPAAPFIRTVKEISNYIAPEVTRWQAEALQALQEAAEDYLIQLFEDSMLCAIHAKRVTLMKKDWELARRLGKKGQPW

SEQ ID NO: 70 - protein, 96 aa, Artificial Sequence
  Description of Artificial Sequence: Synthetic polypeptide
  Helianthus exilis serpentine sunflower CENH3 histone domain
PGTQALREIRRLQKTVELIIPAAPFIRTVKEISNYMAPEITRWQAEALQALQEAAEDYLIQLFEDSMLCAIHAKRVTLMKKDWELARRIGKKGQPW

SEQ ID NO: 71 - protein, 95 aa, Artificial Sequence
  Description of Artificial Sequence: Synthetic polypeptide
  Gossypium hirsutum upland cotton CENH3 histone domain
AGTRALQEIRKYQKTSNLLVPAASFIREVRAISYRFAPDINRWQAEALVAIQEAEDYLIQLFGDAMLCAIHAKRVTLMKKDIQLARRLGGMGQPW

SEQ ID NO: 72 - protein, 93 aa, Artificial Sequence
  Description of Artificial Sequence: Synthetic polypeptide
  Glycine max soybean CENH3 histone domain
SGTVALREIRHFQRSCELLIPAAPFIRCVKQITNQFSTEVSRWTPEAVVALQEAAEEYLVHLFEDGMLCAIHARRITLMKKDIELARRLGGIG

SEQ ID NO: 73 - protein, 96 aa, Artificial Sequence
  Description of Artificial Sequence: Synthetic polypeptide
  Cucumis melo cantaloupe CENH3 histone domain
PGTVALREIRNLQKSWNLLIPASCFIRAVKEVSNQLAPQITRWQAEALVALQEAAEDFLVHLFEDTMLCAIHAKRVTIMKKDFELARRLGGKGRPW

SEQ ID NO: 74 - protein, 96 aa, Artificial Sequence
  Description of Artificial Sequence: Synthetic polypeptide
  Solanum chacoense Chaco potato CENH3 histone domain
PGSVALREIRHFQKTWNLVIPAAPFIRLVREISHFFAPGVTRWQAEALIAIQEAAEDFLVHLFEDAMLCAIHAKRVTLMKKDFELARRLGGKGQPW

SEQ ID NO: 75 - protein, 96 aa, Artificial Sequence
  Description of Artificial Sequence: Synthetic polypeptide
  Solanum lycopersicum tomato CENH3 histone domain
PGTVALREIRHFQKTWDLLIPAAPFIRLVREISHFYAPGVTRWQAEALIAIQEAAEDFLVHLFEDAMLCAIHAKRVTLMKKDFELARRLGGKGQPW

SEQ ID NO: 76 - protein, 96 aa, Artificial Sequence
  Description of Artificial Sequence: Synthetic polypeptide
  Nicotiana tabacum allotetraploid tobacco CENH3-1 histone domain
PGTVALREIRRFQKTWDLLIPAAPFIRLVKEISHFFAPEVTRWQAEALIALQEAAEDFLVHLFDDSMLCAIHAKRVTLMKKDFELARRLGGKARPW

SEQ ID NO: 77 - protein, 96 aa, Artificial Sequence
  Description of Artificial Sequence: Synthetic polypeptide
  Nicotiana tabacum allotetraploid tobacco CENH3-2 histone domain
PGTVALREIRRFQKTWNLLIPAAPFIRLVKEISYFFAPEVTRWQAEALIALQEAAEDFLVHLFDDSMLCAIHAKRVTLMKKDFELARRLGGKARPW

SEQ ID NO: 78 - protein, 96 aa, Artificial Sequence; identical to SEQ ID NO: 76
  Description of Artificial Sequence: Synthetic polypeptide
  Nicotiana tomentosiformis diploid tobacco CENH3 histone domain

SEQ ID NO: 79 - protein, 96 aa, Artificial Sequence
  Description of Artificial Sequence: Synthetic polypeptide
  Vitis vinifera European wine grape CENH3 histone domain
PGTVALREIRRFQKTTHLLIPAAPFIRTVREISYFFAPEISRWTAEALVALQEAAEDYLVHLFEDAMLCAIHAKRVTLMKKDWELARRIGGKGQPW

SEQ ID NO: 80 - protein, 96 aa, Artificial Sequence; identical to SEQ ID NO: 77
  Description of Artificial Sequence: Synthetic polypeptide
  Nicotiana sylvestris woodland tobacco CENH3 histone domain

SEQ ID NO: 81 - protein, 97 aa, Artificial Sequence
  Description of Artificial Sequence: Synthetic polypeptide
  Crucihimalaya himalaica Himalayan rockcress CENH3 histone domain
AGTVALREIRHFQKNTNLLIPAASFIRQVKSITYAVAPPQITRWTAEALVALQEAAEDYLVGLFSDSMLCAIHARRVTLMRKDFELARRLGGKGRPW

SEQ ID NO: 82 - protein, 97 aa, Artificial Sequence
  Description of Artificial Sequence: Synthetic polypeptide
  Arabidopsis lyrata lyre-leaved rockcress CENH3 histone domain
PGTVALREIRHFQKQTNLLIPAASFIRQVRSITHALAPPQINRWTAEALVALQEAAEDYLVGLFSDSMLCAIHARRVTLMRKDFELARRLGGKGRPW

SEQ ID NO: 83 - protein, 90 aa, Artificial Sequence
  Description of Artificial Sequence: Synthetic polypeptide
  Capsella bursapastoris shepherd′s purse CENH3 histone domain
PGTVALREIRHYQKGTSLLIPAAAFIRQVRSITNAVAPREVNRWTAEALVALQEAAEDFLVGLFSDSMLCAIHARRVTLMRKDFDLARRL

SEQ ID NO: 84 - protein, 97 aa, Artificial Sequence
  Description of Artificial Sequence: Synthetic polypeptide
  Raphanus sativus radish CENH3 histone domain
PGTVALREIRHFQKSTKLLIPSAPFIREVRSITHNLAAAYVTRWTAEALIALQEAAEDFLVGLFSDAMLCAIHAKRVTLMRKDFELARRLGGKGRPF

SEQ ID NO: 85 - protein, 97 aa, Artificial Sequence
  Description of Artificial Sequence: Synthetic polypeptide
  Eruca sativa arugula CENH3 histone domain
PGTVALREIRHFQKTTKLLIPAATFIRLVRSITLDRAKPQVTRWTAEALVALQEAAEDYLVGLFSDSMLCAIHAKRVTLMRKDFELARRLGGKGRPW

SEQ ID NO: 86 - protein, 97 aa, Artificial Sequence
  Description of Artificial Sequence: Synthetic polypeptide
  Olimarabidopsis pumila dwarf rocket CENH3-1 histone domain
PGTVALREIRHFQKTTSLLLPAAPFIRQVRSISSALAPREITRWTAEALVALQEAAEDYLVGLFSDSMLCAIHAKRVTLMRKDFELARRLGGKGRPW

SEQ ID NO: 87 - protein, 97 aa, Artificial Sequence
  Description of Artificial Sequence: Synthetic polypeptide
  Olimarabidopsis pumila dwarf rocket CENH3-2 histone domain
PGTVALREIRHFQKTTSFLIPAAPFIRQVRSISSALAPTQITRWTAEALVALQEAAEDYLVGLFSDSMLCAIHAKRVTLMRKDFELARRLGGKGRPW

SEQ ID NO: 88 - protein, 97 aa, Artificial Sequence
  Description of Artificial Sequence: Synthetic polypeptide
  Turritis glabra tower mustard CENH3 histone domain
PGTIALREIRYFQKNTNLLIPAASFIREVRSITHALAPPQISRWTAEALVALQEAAEDYLVGLFSDSMLCAIHARRVTLMRKDFELARRIGGKGRPW

SEQ ID NO: 89 - protein, 97 aa, Artificial Sequence
  Description of Artificial Sequence: Synthetic polypeptide
  Arabidopsis halleri CENH3-1 histone domain
PGTVALREIRHFQKQTNLLIPAASFIRQVRSITHALAPPQINRWTAEALVALQEAAEDYLVGLFSDSMLCAIHARRVTLMRKDFELTRRLGGKGRPW

SEQ ID NO: 90 - protein, 97 aa, Artificial Sequence; identical to SEQ ID NO: 82
  Description of Artificial Sequence: Synthetic polypeptide
  Arabidopsis halleri CENH3-2 histone domain

SEQ ID NO: 91 - protein, 97 aa, Artificial Sequence
  Description of Artificial Sequence: Synthetic polypeptide
  Arabidopsis lyrata CENH3 HTR12A histone domain
PGTVALREIRHFQKQTNLLIPAASFIRQARSITHALAPPQINRWTAEALVALQEAAEDYLVGLFSDSMLCAIHARRVTLMRKDFELARRLGGKGRPW

SEQ ID NO: 92 - protein, 97 aa, Artificial Sequence; identical to SEQ ID NO: 82
  Description of Artificial Sequence: Synthetic polypeptide
  Arabidopsis lyrata CENH3 HTR12B histone domain

SEQ ID NO: 93 - protein, 103 aa, Artificial Sequence
  Description of Artificial Sequence: Synthetic polypeptide
  Saccharum officinalis sugarcane CENH3 histone domain
VGTVALREIRKYQKSTEPLIPFAPFVRVVKELTGFITDWRIGRYTPEALLALQEAAEFHLIELFQVANLCAIHAKRVTVMQKDIQLARRIGGKRWAYPFFLPY

SEQ ID NO: 94 - protein, 48 aa, Artificial Sequence
  Description of Artificial Sequence: Synthetic polypeptide
  Brassica napa turnip CENH3 histone domain
PGTVALREIRHFQKTTKLLIPAASFIREVRSVTQIFAPPDVTRWTAEA

SEQ ID NO: 95 - protein, 91 aa, Artificial Sequence
  Description of Artificial Sequence: Synthetic polypeptide
  Physcomitrella patens moss CENH3 histone domain
PGTKALQEIRHYQKTCDLLIPRLPFARYVKEITMMYASDVSRWTAEALTALQEATEDYMCHLFEDTNLCAIHAKRVTIMPKDLQLARRLRG

SEQ ID NO: 96 - protein, 98 aa, Artificial Sequence
  Description of Artificial Sequence: Synthetic polypeptide
  Pinus taeda loblolly pine CENH3 histone domain
PGTVALREIKRYQKSFELLIPSLPFARIVRELTMYYSQVVSRWAAEALVALQEAAEDYIVHLFEDTNLCAIHAKRVTIMPRDLRLARRLRGGGLDRPW

SEQ ID NO: 97 - protein, 97 aa, Artificial Sequence
  Description of Artificial Sequence: Synthetic polypeptide
  Boechera holboellii Holboell′s rockcress CENH3 histone domain
PGTVALREIRYFQKSINLLIPAASFIRQVRSITHALAPPQITRWTAEALVALQEAAEDYLVGLFSDSMLCAIHARRVTLMRKDFELARRLGGKGRPW

SEQ ID NO: 98 - protein, 97 aa, Artificial Sequence
  Description of Artificial Sequence: Synthetic polypeptide
  Boechera stricta Drummond′s rockcress CENH3 histone domain
PGTVALREIRHFQKSINLLIPAASFIRQVRSITHALAPPQITRWTAEALVALQEAAEDYLVGLFSDSMLCAIHARRITLMRKDFELARRLGGKGRPW

SEQ ID NO: 99 - protein, 97 aa, Artificial Sequence
  Description of Artificial Sequence: Synthetic polypeptide
  Lepidum virginicum Virginia pepperweed CENH3 histone domain
PGTVALREIRHFQKSTHLLIPAAAFIREVRCITQAVAPPQISRWTAEALVALQEAAEDYVVGLLSDSMLCAIHARRVTLMRKDFELARRLGGKGRPW

SEQ ID NO: 100 - protein, 146 aa, Artificial Sequence
  Description of Artificial Sequence: Synthetic polypeptide
  Cardamine flexuosa woodland bittercress CENH3 histone domain
PGTVALREIRHFQKSTNLLIPAASFIRQVRSITQMYAPPQINRWTAEALVALQEAAEDYLVGLFSDSMLCAIHARRVTLMRKDFELARRLGGKGRPWLMAIQEAAEDFLVGLFSDAMLCAIHARRVTLMRKDFELARRLGGKGRPL

SEQ ID NO: 101 - protein, 142 aa, Artificial Sequence
  Description of Artificial Sequence: Synthetic polypeptide
MARTKHLAKRSRTTSAAPSATPSTPSRKSPRSAPATSVQKPKQKKRYRPVALREIRHFQKTWDLLIPAAPFIRLVREISHFYAPGVTRWQAEALIAIQEAAEDFLVHLFEDAMLCAIHAKRVTLMKKDFELARRLGGKGQPW

SEQ ID NO: 102-109 - skipped (8 entries)

SEQ ID NO: 110 - protein, 140 aa, Artificial Sequence
  Description of Artificial Sequence: Synthetic polypeptide
MARTKHLAKRSRTTSAAPSATPSTPSRKSPRSAPATSVQKPKQKKRSVALREIRHFQKTWDLLIPAAPFIRLVREISHFYAPGVTRWQAEALIAIQEAAEDFLVHLFEDAMLCAIHAKRVTLMKKDFELARRLGGKGQPW

SEQ ID NO: 111-115 - skipped (5 entries)

SEQ ID NO: 116 - protein, 176 aa, Artificial Sequence
  Description of Artificial Sequence: Synthetic polypeptide
MARTKHRVTRSQPRNQTDAAGASSSQAAGPTTTPTRRGGEGGDNTQQTNPTTSPATGTRRGAKRSRQAMPRGSQKKSYRYRPVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 117 - protein, 167 aa, Artificial Sequence
  Description of Artificial Sequence: Synthetic polypeptide
MARTKHRVTRSQPRNQTDAAGASSSQAAGPTTTPTRRGGEGGDNTQQTNPTTSPATGTRRGAKRSRQAMPRGSQKKSYRYRPGKQTNLLIPAASFIREVRSITHMLAPPQINRWTAEALVALQEAAEDYLVGLFSDSMLCAIHARRVTLMRKDFELARRLGGKGRPW

SEQ ID NO: 118-125 - skipped (8 entries)

SEQ ID NO: 126 - protein, 145 aa, Artificial Sequence
  Description of Artificial Sequence: Synthetic polypeptide
MARTKHRVTRSQPRNQTDAAGASSSQAAGPTTRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 127 - protein, 186 aa, Artificial Sequence
  Description of Artificial Sequence: Synthetic polypeptide
MARTKHRVTRSQPRNQTDAAGASSSQAAGPTAGPISNLKFTPTRRGGEGGDNTQQTNPTTSPATGTRRGAKRSRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 128 - protein, 181 aa, Artificial Sequence
  Description of Artificial Sequence: Synthetic polypeptide
MARTKHRVTRSQPRNQTDAAGASSSQAAGPTTKLKTPTRRGGEGGDNTQQTNPTTSPATGTRRGAKRSRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 129 - protein, 180 aa, Artificial Sequence
  Description of Artificial Sequence: Synthetic polypeptide
MARTKHRVTRSQPRNQTDAAGASSSQAAGPTIELTPTRRGGEGGDNTQQTNPTTSPATGTRRGAKRSRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 130 - protein, 179 aa, Artificial Sequence
  Description of Artificial Sequence: Synthetic polypeptide
MARTKHRVTRSQPRNQTDAAGASSSQAAGPTTTPTRRGGEGGDNTQQTNPTTSPATGTRRGAKRSTRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 131 - protein, 141 aa, Artificial Sequence
  Description of Artificial Sequence: Synthetic polypeptide
MARTKHRVTRSQPRNQTDAAGASSSQAAGPTMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 132 - protein, 185 aa, Artificial Sequence
  Description of Artificial Sequence: Synthetic polypeptide
MARTKHRVTRSQPRNQTDAAGASSSQAAGPTIVMFLPFSTPTRRGGEGGDNTQQTNPTTSPATGTRRGAKRSRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 133 - protein, 145 aa, Artificial Sequence; identical to SEQ ID NO: 126
  Description of Artificial Sequence: Synthetic polypeptide

SEQ ID NO: 134 - protein, 176 aa, Artificial Sequence
  Description of Artificial Sequence: Synthetic polypeptide
MARTKHRVTRSQPRNQTDAAGASSSQAAGPTTTPTRRGGEGGDNTQQTNPTTSPATGTRRGAKRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 135 - protein, 124 aa, Artificial Sequence
  Description of Artificial Sequence: Synthetic polypeptide
MARTKHRVTRSQPRNQTGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 136 - protein, 175 aa, Artificial Sequence
  Description of Artificial Sequence: Synthetic polypeptide
MARTKHRVTRSQPRNQTDAAGASSSQAAGPTTTPTRRGGEGGDNTQQTNPTTSPATGTRRGAKRSRQAMPRGSQKKSMPGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 137 - protein, 180 aa, Artificial Sequence
  Description of Artificial Sequence: Synthetic polypeptide
MARTKHRVTRSQPRNQTDAAGASSSQAAGPTTTPTRRGGEGGDNTQQTNPTTSPATGTRRGAKRSRQAMPRGSQKKSYRYRPGTVALKEIRHCVIKKQTNLLIPAASFIREVRSITHMLAPPQINRWTAEALVALQEAAEDYLVGLFSDSMLCAIHARRVTLMRKDFELARRLGGKGRPW

SEQ ID NO: 138 - protein, 177 aa, Artificial Sequence
  Description of Artificial Sequence: Synthetic polypeptide
MARTKHRVTRSQPRNQTDAAGASSSQAAGPTTTPTRRGGEGGDNTQQTNPTTSPATGTRRGAKRSRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGGRPW

SEQ ID NO: 139 - protein, 175 aa, Artificial Sequence
  Description of Artificial Sequence: Synthetic polypeptide
MARTKHRVTRSQPRNQTDAAGASSSQAAGPTTTPTRRGGEGGDNTQQTNPTTSPATGTRRGAKRSRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGPW

SEQ ID NO: 140 - protein, 186 aa, Artificial Sequence
  Description of Artificial Sequence: Synthetic polypeptide
MARTKHRVTRSQPRNQTDAAGASSSQAAGPTTTPTRRGGEGGDNTQQTNPTTSPATGTRRGAKRSRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGDRKLTHYSHLLHCK

SEQ ID NO: 141 - protein, 177 aa, Artificial Sequence
  Description of Artificial Sequence: Synthetic polypeptide
MARTKHRVTRSQPRNQTDAAGASSSQAAGPTTTPTRRGGEGGDNTQQTNPTTSPATGTRRGAKRSRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGKGRPW

SEQ ID NO: 142 - protein, 173 aa, Artificial Sequence
  Description of Artificial Sequence: Synthetic polypeptide
MARTKHRVTRSQPRNQTDAAGASSSQAAGPTTTPTRRGGEGGDNTQQTNPTTSPATGTRRGAKRSRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGG

SEQ ID NO: 143 - protein, 141 aa, Artificial Sequence
  Description of Artificial Sequence: Synthetic polypeptide
MARTKHLAKRSRTTSAAPSATPSTPSRKSPRSAPATSVQKPKQKKRYTVALREIRHFQKTWDLLIPAAPFIRLVREISHFYAPGVTRWQAEALIAIQEAAEDFLVHLFEDAMLCAIHAKRVTLMKKDFELARRLGGKGQPW

SEQ ID NO: 144 - protein, 175 aa, Artificial Sequence
  Description of Artificial Sequence: Synthetic polypeptide
MARTKHRVTRSQPRNQTDAAGASSSQAAGPTTRRGGEGGDNTQQTNPTTSPATGTRRGAKRSRQAMPRGSQKKSYRYRPGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 145 - protein, 11 aa, Unknown
  Description of Unknown: CENH3 sequence
TVALKEIRHFQ

SEQ ID NO: 146 - protein, 6 aa, Unknown
  Description of Unknown: CENH3 sequence
PGTVAL

SEQ ID NO: 147 - protein, 9 aa, Artificial Sequence
  Description of Artificial Sequence: Synthetic peptide
RYRPGTVAL

SEQ ID NO: 148 - protein, 7 aa, Artificial Sequence
  Description of Artificial Sequence: Synthetic peptide
RYRPVAL

SEQ ID NO: 149 - protein, 11 aa, Artificial Sequence
  Description of Artificial Sequence: Synthetic peptide
KKRYRPGTVAL

SEQ ID NO: 150 - protein, 7 aa, Artificial Sequence
  Description of Artificial Sequence: Synthetic peptide
KKRSVAL

SEQ ID NO: 151 - protein, 7 aa, Artificial Sequence
  Description of Artificial Sequence: Synthetic peptide
GPTTTPT

SEQ ID NO: 152 - protein, 15 aa, Artificial Sequence
  Description of Artificial Sequence: Synthetic peptide
GPTAGPISNLKFTPT

SEQ ID NO: 153 - protein, 9 aa, Artificial Sequence
  Description of Artificial Sequence: Synthetic peptide
GPTTTPTRR

SEQ ID NO: 154 - protein, 6 aa, Artificial Sequence
  Description of Artificial Sequence: Synthetic peptide
GPTTRR

SEQ ID NO: 155 - protein, 10 aa, Artificial Sequence
  Description of Artificial Sequence: Synthetic peptide
GPTTKLKTPT

SEQ ID NO: 156 - protein, 7 aa, Artificial Sequence
  Description of Artificial Sequence: Synthetic peptide
GPTTTPT

SEQ ID NO: 157 - protein, 9 aa, Artificial Sequence
  Description of Artificial Sequence: Synthetic peptide
GPTIELTPT

SEQ ID NO: 158 - protein, 6 aa, Artificial Sequence
  Description of Artificial Sequence: Synthetic peptide
KRSRQA

SEQ ID NO: 159 - protein, 7 aa, Artificial Sequence
  Description of Artificial Sequence: Synthetic peptide
KRSTRQA

SEQ ID NO: 160 - protein, 43 aa, Artificial Sequence
  Description of Artificial Sequence: Synthetic polypeptide
GPTTTPTRRGGEGGDNTQQTNPTTSPATGTRRGAKRSRQAMPR

SEQ ID NO: 161 - protein, 6 aa, Artificial Sequence
  Description of Artificial Sequence: Synthetic peptide
GPTMPR

SEQ ID NO: 162 - protein, 40 aa, Artificial Sequence
  Description of Artificial Sequence: Synthetic polypeptide
GPTTTPTRRGGEGGDNTQQTNPTTSPATGTRRGAKRSRQA

SEQ ID NO: 163 - protein, 7 aa, Artificial Sequence
  Description of Artificial Sequence: Synthetic peptide
GPTTRQA

SEQ ID NO: 164 - protein, 8 aa, Artificial Sequence
  Description of Artificial Sequence: Synthetic peptide
AKRSRQAM

SEQ ID NO: 165 - protein, 6 aa, Artificial Sequence
  Description of Artificial Sequence: Synthetic peptide
AKRQAM

SEQ ID NO: 166 - protein, 61 aa, Artificial Sequence
  Description of Artificial Sequence: Synthetic polypeptide
RNQTDAAGASSSQAAGPTTTPTRRGGEGGDNTQQTNPTTSPATGTRRGAKRSRQAMPRGSQ

SEQ ID NO: 167 - protein, 7 aa, Artificial Sequence
  Description of Artificial Sequence: Synthetic peptide
RNQTGSQ

SEQ ID NO: 168 - protein, 10 aa, Artificial Sequence
  Description of Artificial Sequence: Synthetic peptide
KKSYRYRPGT

SEQ ID NO: 169 - protein, 7 aa, Artificial Sequence
  Description of Artificial Sequence: Synthetic peptide
KKSMPGT

SEQ ID NO: 170 - protein, 11 aa, Artificial Sequence
  Description of Artificial Sequence: Synthetic peptide
EIRHFQKQTNL

SEQ ID NO: 171 - protein, 13 aa, Artificial Sequence
  Description of Artificial Sequence: Synthetic peptide
EIRHCVIKKQTNL

SEQ ID NO: 172 - protein, 7 aa, Artificial Sequence
  Description of Artificial Sequence: Synthetic peptide
GGKGRPW

SEQ ID NO: 173 - protein, 6 aa, Artificial Sequence
  Description of Artificial Sequence: Synthetic peptide
GGGRPW

SEQ ID NO: 174 - protein, 7 aa, Artificial Sequence
  Description of Artificial Sequence: Synthetic peptide
GGKGRPW

SEQ ID NO: 175 - protein, 4 aa, Artificial Sequence
  Description of Artificial Sequence: Synthetic peptide
GGPW

SEQ ID NO: 176 - protein, 9 aa, Artificial Sequence
  Description of Artificial Sequence: Synthetic peptide
RLGGKGRPW

SEQ ID NO: 177 - protein, 17 aa, Artificial Sequence
  Description of Artificial Sequence: Synthetic peptide
RLGDRKLTHYSHLLHCK

SEQ ID NO: 178 - protein, 7 aa, Artificial Sequence
  Description of Artificial Sequence: Synthetic peptide
GGKGRPW

SEQ ID NO: 179 - protein, 6 aa, Artificial Sequence
  Description of Artificial Sequence: Synthetic peptide
GKGRPW

SEQ ID NO: 180 - protein, 7 aa, Artificial Sequence
  Description of Artificial Sequence: Synthetic peptide
GGKGRPW

SEQ ID NO: 181 - protein, 9 aa, Artificial Sequence
  Description of Artificial Sequence: Synthetic peptide
RYRPGTVAL

SEQ ID NO: 182 - protein, 6 aa, Artificial Sequence
  Description of Artificial Sequence: Synthetic peptide
RYTVAL

SEQ ID NO: 183 - protein, 101 aa, Homo sapiens
HSRRRQGWLKEIRKLQKSTHLLIRKLPFSRLAREICVKFTRGVDFNWQAQALLALQEAAEAFLVHLFEDAYLLTLHAGRVTLFPKDVQLARRIRGLEEGLG

SEQ ID NO: 184 - protein, 100 aa, Mus musculus
RRQKFMWLKEIKTLQKSTDLLFRKKPFSMVVREICEKFSRGVDFWWQAQALLALQEAAEAFLIHLFEDAYLLSLHAGRVTLFPKDIQLTRRIRGFEGGLP

SEQ ID NO: 185 - protein, 100 aa, Rattus norvegicus
RRRRFLWLKEIKNLQKSTDLLFRKKPFGLVVREICGKFSRGVDLYWQAQALLALQEAAEAFLVHLFEDAYLLSLHAGRVTLFPKDVQLARRIRGIEGGLG

SEQ ID NO: 186 - protein, 101 aa, Gallus gallus
RYRPGQRALREIRRYQSSTALLLRRQPFARVVREICLLFTRGVDYRWQAMALLALQEAAEAFLVHLLEDAYLCSLHARRVTLYPKDLQLARRLRGLQGEGF

SEQ ID NO: 187 - protein, 101 aa, Xenopus laevis
RFRPGTRALMEIRKYQKSTELLIRKAPFSRLVREVCMTYACGMNYNWQSMALMALQEASEAFLVRLFEDSYLCSLHAKRVTLYVQDIQLARRIRGVNEGLG

SEQ ID NO: 188 - protein, 98 aa, Danio rerio
KFRPGTRALMEIRKYQKSTGLLLRKAPFSRLVREVCQMFSREHMMWQGYALMALQEAAEAFMVRLFSDANLCAIHAKRVTLFPRDIQLARRIRGVEHM

SEQ ID NO: 189 - protein, 100 aa, Drosophila melanogaster
PMSRAKRMDREIRRLQHHPGTLIPKLPFSRLVREFIVKYSDDEPLRVTEGALLAMQESCEMYLTQRLADSYMLTKHRNRVTLEVRDMALMAYICDRGRQF

SEQ ID NO: 190 - protein, 99 aa, Caenorhabditis elegans
RYRPGQKALEEIRKYQKTEDLLIQKAPFARLVREIMQTSTPFGADCRIRSDAISALQEAAEAFLVEMFEGSSLISTHAKRVTLMTTDIQLYRRLCLRHL

SEQ ID NO: 191 - protein, 100 aa, Schizosaccharomyces pombe
RYRPGTTALREIRKYQRSTDLLIQRLPFSRIVREISSEFVANFSTDVGLRWQSTALQCLQEAAEAFLVHLFEDTNLCAIHAKRVTIMQRDMQLARRIRGA

SEQ ID NO: 192 - protein, 101 aa, Candida albicans
RYRPGTKALREIRQYQKSTDLLIRKLPFARLVREISLDFVGPSYGLRWQSNAILALQEASESFLIHLLEDTNLCAIHAKRVTIMQKDIQLARRIRGQSWIL

SEQ ID NO: 193 - protein, 99 aa, Saccharomyces cerevisiae
KYTPSELALYEIRKYQRSTDLLISKIPFARLVKEVTDEFTTKDQDLRWQSMAIMALQEASEAYLVGLLEHTNLLALHAKRITIMKKDMQLARRIRGQFI

SEQ ID NO: 194 - protein, 100 aa, Arabidopsis thaliana
RYRPGTVALKEIRHFQKQTNLLIPAASFIREVRSITHMLAPPQINRWTAEALVALQEAAEDYLVGLFSDSMLCAIHARRVTLMRKDFELARRLGGKGRPW

SEQ ID NO: 195 - protein, 96 aa, Homo sapiens
RYRPGTVALREIRRYQKSTELLIRKLPFQRLVREIAQDFKTDLRFQSSAVMALQEACEATLVGLFEDTNLCAIHAKRVTIMPKDIQLARRIRGERA